SEQUENCE LISTING (2 sequences)

SEQ ID NO: 1 - protein, 25 aa, artificial sequence
  ATI Tail 1
GRGRGRAAAAATAATAAAAAAATAA

SEQ ID NO: 2 - protein, 24 aa, artificial sequence
  ATI Tail 2
GRGRGRAATAAAAAATAATATTAT

(19) United States
(12) Patent Application Publication
(10) Pub. No.: US 2022/0389067 A1
(43) Pub. Date: Dec. 8, 2022

(54) Methods of Treating Neurodegenerative Diseases Caused by G4C2 Expansion in C9ORF72

(71) Applicant: The Board of Trustees of the Leland Stanford Junior University, Stanford, CA (US)

(72) Inventors: Bingwei Lu, Stanford, CA (US); Zhihao Wu, Palo Alto, CA (US); Shuangxi Li, Stanford, CA (US)

(73) Assignee: The Board of Trustees of the Leland Stanford Junior University, Stanford, CA (US)

(21) Appl. No.: 17/755,716

(22) PCT Filed: Nov. 5, 2020
(86) PCT No.: PCT/US2020/059100

§ 371 Date: May 5, 2022

Related U.S. Application Data
(60) Provisional application No. 62/931,058, filed on Nov. 5, 2019.

(51) Int. Cl. C07K 14/47; A61P 21/00; A61P 25/14; A61P 25/28

(52) U.S. Cl. CPC C07K 14/4703; A61P 21/00; A61P 25/14; A61P 25/28; A61K 38/00

(57) ABSTRACT

Methods of treating subjects having G4C2 dipeptide repeat expansion in the gene C9ORF72, including subjects having amyotrophic lateral sclerosis or frontotemporal degeneration (ALS/FTD), are provided. Compounds directed at reducing the toxicity of G4C2 dipeptide repeat expansion in the gene C9ORF72 are described.

### CROSS REFERENCE TO RELATED APPLICATIONS

This application claims priority to U.S. Provisional Application Ser. No. 62/931,068, entitled “Methods of Treating Related Neurodegenerative Diseases Caused by G4C2 Expansion in C9ORF72,” filed Nov. 5, 2019, which is incorporated herein by reference in its entirety.

### STATEMENT REGARDING FEDERALLY SPONSORED RESEARCH OR DEVELOPMENT

This invention was made with Government support under contracts MH080378 and NS084412 awarded by the National Institutes of Health. The Government has certain rights in the invention.

### SEQUENCE LISTING

This application includes material in an electronic Sequence Listing. The material in the electronic Sequence Listing is submitted as a text (.txt) file entitled “05099PCT_SeqList_ST25.txt” created on Feb. 2, 2021, which has a file size of approximately 1 KB. The electronic Sequence Listing is hereby incorporated by reference in its entirety.

### TECHNICAL FIELD

The disclosure relates to methods of treatment, and more particularly to pharmaceutical formulations and methods to treat ALS/FTD.

### BACKGROUND

Amyotrophic lateral sclerosis (ALS) is characterized primarily by the selective loss of upper motor neurons (MNs) in the motor cortex and lower MNs in the brainstem and spinal cord. Traditionally considered a MN disease, ALS is emerging as a “multisystem” disease with pathological changes in various cell types including neurons, as well as glia and muscle cells. Myopathic features are common in ALS.

Expansion of G4C2 repeat in C9ORF72 is the most common genetic cause of ALS and frontotemporal degeneration (FTD) (ALS/FTD), accounting for ˜40% of familial and 5-10% of sporadic ALS cases. A number of mechanisms have been put forward to explain the pathogenesis of G4C2 repeat expansion. Increasing evidences emphasize the contribution of dipeptide repeats (DPRs) translated from G4C2 repeat-carrying transcripts by an unconventional, repeat-associated non-AUG (RAN) translation process.

Expansion of G4C2 repeat in C9ORF72 is also indicated in other neurodegenerative diseases, including Alzheimer's disease and Parkinson's disease. It is likely that the other forms of neurodegeneration arise from G4C2 repeat expansion.

### SUMMARY

Various embodiments are directed to pharmaceutical formulations for and treatments of neurodegeneration disorders caused by G4C2 expansion in the gene C9ORF72. In various embodiments, a subject having G4C2 expansion in the gene C9ORF72 is treated with a compound. In various embodiments, a subject having ALS/FTD is treated with a compound. In various embodiments, a compound is an agonist of AKT, an agonist of Notch, an agonist of YME1L, an antagonist of CAT-tailing, a mitochondrial uncoupler, or a potassium ion-proton antiporter.

In an embodiment, a subject having expansion of G4C2 repeats in C9ORF72 is treated. The subject is administered a noncanonical Notch signaling agonist, an AKT agonist, a YME1L agonist, an inhibitor of CAT-tailing, a mitochondrial uncoupler, or a potassium ion-proton antiporter.

In another embodiment, the subject is a human, an animal model, a cell line model, an organoid model, or a tissue model.

In yet another embodiment, the subject is a human having amyotrophic lateral sclerosis or frontotemporal degeneration.

In a further embodiment, the subject is a human having Alzheimer's disease or Parkinson's disease.

In still yet another embodiment, the subject is administered a noncanonical Notch signaling agonist or an AKT agonist selected from: SC79, kinetin, kinetin triphosphate, kinetin monophosphate, kinetin riboside, or phosphatidylinositol (3,4,5)-triphosphate (PIP3).

In yet a further embodiment, the subject is administered a YME1L agonist.

In an even further embodiment, the subject is administered a CAT-tailing inhibitor selected from: anisomycin, puromycin, neomycin, borrelidin, or alanyl-AMP.

In yet an even further embodiment, the subject is administered a mitochondrial uncoupler selected from: 2, 4-dinitrophenol (DNP); DNP-methylethyl (DNPME), diazoxide, Carbonyl cyanide 4-(trifluoromethoxy) phenylhydrazone (FCCP), Carbonyl cyanide 3-chlorophenylhydrazone (CCCP), 4-[(E)-2-(5,6,7,8-Tetrahydro-5,5,8,8-tetramethyl-2-naphthalenyl)-1-propenyl]benzoic acid (TTNPB), valinomycin; N5,N6-bis(2-Fluorophenyl)-oxadiazolo-pyrazine-5,6-diamine (BAM15), controlled-released mitochondrial protonophore (CRMP), Rhodamine 19 butyl ester (C4R1), Dodecyltriphenylphosphonium (C12TPP), niclosamide ethanolamine salt (NEN), or 10-(6′-plastoquinonyl) decyltriphenyl-phosphonium) (SkQ1).

In still yet an even further embodiment, the subject is administered a K⁺/H⁺ antiporter selected from: nigericin, monensin, narasin, salinomycin, lenoremycin, septamycin, carriomycin, X-14766A, noboritomycin A, alborixin, ionomycin, A23187, X=14547A, lysocellin, or lasalocid.

In an embodiment, a subject having expansion of G4C2 repeats in C9ORF72 is treated. The subject is administered a genetic modifier to increase noncanonical Notch signaling agonist.

In another embodiment, the genetic modifier increases expression of Notch, PINK1, mTORC2, or AKT.

In an embodiment, a subject having expansion of G4C2 repeats in C9ORF72 is treated. The subject is administered a genetic modifier to increase YME1L expression.

In an embodiment, a subject having expansion of G4C2 repeats in C9ORF72 is treated. The subject is administered a genetic modifier to decrease activity of CAT-tailing.

In another embodiment, the genetic modifier downregulates expression of NEMF, AARS, TARS, IARS, ZNF598 or Rack1.

In yet another embodiment, the genetic modifier upregulates expression of USP10, ANKZF1, VCP, Pelo, Ltn1 and ABCE1.

In an embodiment, a subject having expansion of G4C2 repeats in C9ORF72 is treated. The subject is administered a genetic modifier to decrease activity of MICOS.

In another embodiment, the genetic modifier downregulates expression of Mic60, Mic19, Mic10, Mic27, Opa1, or Tom40.

In an embodiment, a subject having expansion of G4C2 repeats in C9ORF72 is treated. The subject is administered a genetic modifier to increase activity of a potassium ion-proton antiporter.

In another embodiment, the genetic modifier increases expression of LETM1.

In yet another embodiment, the subject is a human, an animal model, a cell line model, an organoid model, or a tissue model.

### BRIEF DESCRIPTION OF THE DRAWINGS

The description will be more fully understood with reference to the following figures, which are presented as exemplary embodiments of the invention and should not be construed as a complete recitation of the scope of the invention, wherein:

FIG. 1 provides a western blot showing activity of various drugs on protein expression levels of poly(GR) dipeptide repeats in accordance with embodiments.

FIGS. 2 to 11 provide data graphs and microscope imaging results that show mitochondrial defects in GR80 transgenic fly muscle, utilized to support various embodiments.

FIGS. 12 to 28 provide data graphs, microscope imaging results, and biochemical blot results that show mitochondrial localization and Opa1 interaction of GR80, utilized to support various embodiments.

FIGS. 29 to 45 provide data graphs, microscope imaging results, and biochemical blot results that show the effect of GR80 on Mitochondrial Contact Site and Cristae Organizing System (MICOS) and mitochondrial ion homeostasis, utilized to support various embodiments.

FIGS. 46 to 55 provide data graphs, microscope imaging results, and biochemical blot results that show mitochondrial defects in C9-ALS patient fibroblasts and effect of nigericin and genetic manipulation of MICOS components, utilized to support various embodiments.

FIGS. 56 to 62 provide data graphs, microscope imaging results, and biochemical blot results that show YME1L negatively regulates poly(GR) expression and toxicity, utilized to support various embodiments.

FIGS. 63 to 69 provide microscope imaging results, biochemical blot results, and schematic that show stalled translation of poly(GR) on mitochondria and engage of ribosome-associated quality control (RQC) machinery, utilized to support various embodiments.

FIGS. 70 to 83 provide data graphs, microscope imaging results, and biochemical blot results that show RQC and CAT-tailing of ribosome-stalled poly(GR), utilized to support various embodiments.

FIGS. 84 to 99 provide data graphs, microscope imaging results, and biochemical blot results that show negative regulation of poly(GR) expression and toxicity by non-canonical Notch signaling, utilized to support various embodiments.

### DETAILED DESCRIPTION OF THE INVENTION

Turning now to the disclosure and examples, methods of treatment and pharmaceutical formulations for treating neurodegeneration disorders caused by G4C2 expansion in the gene C9ORF72, including amyotrophic lateral sclerosis with frontotemporal degeneration (ALS/FTD), are provided. Various embodiments are directed towards treatments in the clinic for individuals having G4C2 expansion in the gene C9ORF72. Various embodiments are directed towards preclinical treatment assessments in animal models or cell lines having G4C2 expansion in the gene C9ORF72 (or a homolog thereof). Accordingly, various embodiments are directed to administering a treatment to a subject having G4C2 expansion in the gene C9ORF72. It is to be understood that a subject can be a human, an animal model, a cell line model, an organoid model, a tissue model, or other biological system recapitulating G4C2 expansion in the gene C9ORF72 (or a homolog thereof).

Based on recent studies, it is now understood that the gene C9ORF72 can contain a GGGGCC (G4C2) hexanucleotide repeat expansion, which can be expressed into dipeptide repeats and cause mitochondrial toxicity. Specifically, poly(GR) dipeptide repeats are co-translationally imported into mitochondria where it can stabilize, aggregate and cause toxicity.

It is now known that noncanonical Notch signaling via AKT restrains production of poly(GR) dipeptide repeats (see Exemplary Embodiments). Accordingly, agonists of AKT and Notch and genetic means of inducing noncanonical Notch signaling can be utilized in accordance with various embodiments to mitigate toxicity associated with poly(GR) dipeptide repeats. In some embodiments, a subject having G4C2 expansion in the gene C9ORF72 (or a homolog thereof) is administered an agonist of AKT or Notch. Agonists of AKT or Notch include (but are not limited to) SC79, kinetin, kinetin triphosphate, kinetin monophosphate, kinetin riboside, and phosphatidylinositol (3,4,5)-triphosphate (PIP3). In some embodiments, a subject having G4C2 expansion in the gene C9ORF72 (or a homolog thereof) is administered a genetic modulator that enhances noncanonical Notch signaling. Notch signaling can be increased by upregulation of at least one of the following genes: Notch, PINK1, mTORC2, and AKT.

In addition, it is now known that YME1L expression reduces protein expression of poly(GR) dipeptide repeats (see Exemplary Embodiments). Accordingly, agonists of YME1L and genetic means of increasing YME1L expression can be utilized in accordance with various embodiments to mitigate toxicity associated with poly(GR) dipeptide repeats. In some embodiments, a subject having G4C2 expansion in the gene C9ORF72 (or a homolog thereof) is administered an agonist of YME1L. In some embodiments, a subject having G4C2 expansion in the gene C9ORF72 (or a homolog thereof) is administered a genetic modulator that upregulates YME1L expression.

It is further revealed that poly(GR) dipeptide repeats are stabilized by CAT-tailing (i.e., addition of Ala/Thr residues to the C-terminus of proteins) (see Exemplary Embodiments). A number of means to inhibit CAT-tailing can be utilized to destabilize poly(GR) dipeptide repeats such that their expression products can be removed, preventing toxicity. In some embodiments, a subject having G4C2 expansion in the gene C9ORF72 (or a homolog thereof) is administered an antagonist of CAT-tailing. CAT-tailing antagonists include (but are not limited to) anisomycin, puromycin, neomycin, borrelidin, and alanyl-AMP. In some embodiments, a subject having G4C2 expansion in the gene C9ORF72 (or a homolog thereof) is administered a genetic modulator that decreases CAT-tailing activity. CAT-tailing can be decreased by downregulation of at least one the following genes: NEMF, AARS, TARS, IARS, ZNF598 and Rack1, or by the overexpression of at least one of the following genes: USP10, ANKZF1, VCP, Pelo, Ltn1 and ABCE1.

It is also revealed that accumulated poly(GR) dipeptide repeats can enter muscle mitochondria and interact with components of the Mitochondrial Contact Site and Cristae Organizing System (MICOS), altering mitochondrial inner membrane structure and ion homeostasis in ALS/FTD-affected organisms (see Exemplary embodiments). Accordingly, mitochondrial uncouplers can reduce membrane potential to rebalance mitochondrial ion homeostasis and increase health of the mitochondria. In some embodiments, a subject having G4C2 expansion in the gene C9ORF72 (or a homolog thereof) is administered a mitochondrial uncoupler. Mitochondrial uncouplers include (but are not limited to) 2, 4-dinitrophenol (DNP); DNP-methylethyl (DNPME), diazoxide, Carbonyl cyanide 4-(trifluoromethoxy) phenylhydrazone (FCCP), Carbonyl cyanide 3-chlorophenylhydrazone (CCCP), 4-[(E)-2-(5,6,7,8-Tetrahydro-5,5,8,8-tetramethyl-2-naphthalenyl)-1-propenyl]benzoic acid (TTNPB), valinomycin, N5,N6-bis(2-Fluorophenyl)-oxadiazolo-pyrazine-5,6-diamine (BAM15), controlled-released mitochondrial protonophore (CRMP), Rhodamine 19 butyl ester (C4R1), Dodecyltriphenylphosphonium (C12TPP), niclosamide ethanolamine salt (NEN), and 10-(6′-plastoquinonyl) decyltriphenyl-phosphonium) (SkQ1). In some embodiments, a subject having G4C2 expansion in the gene C9ORF72 (or a homolog thereof) is administered a genetic modulator that restores mitochondrial ion homeostasis and health by decreasing MICOS activity. MICOS activity can be decreased by downregulation at least one of the following genes: Mic60, Mic19, Mic10, Mic27, Opa1, and Tom40.

In a similar manner, in order to rebalance mitochondrial potassium ions, a potassium ion-proton (K⁺/H⁺) antiporter can be utilized. In some embodiments, a subject having G4C2 expansion in the gene C9ORF72 (or a homolog thereof) is administered a K⁺/H⁺ antiporter. K⁺/H⁺ antiporters include (but are not limited to) nigericin, monensin, narasin, salinomycin, lenoremycin, septamycin, carriomycin, X-14766A, noboritomycin A, alborixin, ionomycin, A23187, X=14547A, lysocellin, and lasalocid. In some embodiments, a subject having G4C2 expansion in the gene C9ORF72 (or a homolog thereof) is administered a genetic modulator that enhances K⁺/H⁺ antiporter activity. K⁺/H⁺ antiporter activity can be increased by upregulation at least one of the following genes: LETM1.

### Formulations

Provided herein are various embodiments of pharmaceutical compositions which incorporate one or more of certain compounds disclosed herein, or one or more pharmaceutically acceptable salts, prodrugs, or solvates thereof, optionally together with one or more pharmaceutically acceptable carriers thereof and optionally one or more other therapeutic ingredients. Proper formulation is dependent upon the route of administration chosen. Any of the well-known techniques, carriers, and excipients may be used as suitable and as understood in the art. The pharmaceutical compositions disclosed herein may be manufactured in any manner known in the art, e.g., by means of conventional mixing, dissolving, granulating, dragee-making, levigating, emulsifying, encapsulating, entrapping or compression processes. Pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, 21st Edition; Lippincott Williams & Wilkins: Philadelphia, Pa., 2005; Modified-Release Drug Delivery Technology, Rathbone et al, Eds., Drugs and the Pharmaceutical Science, Marcel Dekker, Inc., New York, N.Y., 2002; Vol. 126).

The terms “active ingredient,” “active compound,” and “active substance” refer to a compound, which is administered, alone, in combination with other active compounds, or in combination with one or more pharmaceutically acceptable excipients or carriers, to a subject for treating, preventing, or ameliorating one or more symptoms of a disorder.

The compounds disclosed herein can exist as therapeutically acceptable salts. The term “therapeutically acceptable salt,” as used herein, represents salts or zwitterionic forms of the compounds disclosed herein which are therapeutically acceptable as defined herein. The salts can be prepared during the final isolation and purification of the compounds or separately by reacting the appropriate compound with a suitable acid or base. Therapeutically acceptable salts include acid and basic addition salts. For a more complete discussion of the preparation and selection of salts, refer to “Handbook of Pharmaceutical Salts, Properties, and Use,” Stah and Wermuth, Ed., (Wiley-VCH and VHCA, Zurich, 2002) and Berge et al, J. Pharm. Sci. 1977, 66, 1-19.

Several embodiments incorporate at least one active ingredient for the treatment of a subject having G4C2 expansion in the gene C9ORF72 (or a homolog thereof), which can cause ALS/FTD. In some embodiments, an active ingredient is an agonist of noncanonical Notch signaling or AKT, an agonist of YME1L, an inhibitor of CAT-tailing, a mitochondrial uncoupler, or a K⁺/H⁺ antiporter.

Numerous coating agents can be used in accordance with various embodiments of the invention. In some embodiments, the coating agent is one which acts as a coating agent in conventional delayed release oral formulations, including polymers for enteric coating. Examples include hypromellose phthalate (hydroxy propyl methyl cellulose phthalate; HPMCP); hydroxypropylcellulose (HPC; such as KLUCEL®); ethylcellulose (such as ETHOCEL®); and methacrylic acid and methyl methacrylate (MAA/MMA; such as EUDRAGIT®).

Various embodiments of formulations also include at least one disintegrating agent, as well as diluent. In some embodiments, a disintegrating agent is a super disintegrant agent. One example of a diluent is a bulking agent such as a polyalcohol. In many embodiments, bulking agents and disintegrants are combined, such as, for example, PEARLITOL FLASH®, which is a ready to use mixture of mannitol and maize starch (mannitol/maize starch). In accordance with a number of embodiments, any polyalcohol bulking agent can be used when coupled with a disintegrant or a super disintegrant agent. Additional disintegrating agents include, but are not limited to, agar, calcium carbonate, maize starch, potato starch, tapioca starch, alginic acid, alginates, certain silicates, and sodium carbonate. Suitable super disintegrating agents include, but are not limited to crospovidone, croscarmellose sodium, AMBERLITE (Rohm and Haas, Philadelphia, Pa.), and sodium starch glycolate.

In certain embodiments, diluents are selected from the group consisting of mannitol powder, spray dried mannitol, microcrystalline cellulose, lactose, dicalcium phosphate, tricalcium phosphate, starch, pregelatinized starch, compressible sugars, silicified microcrystalline cellulose, and calcium carbonate.

Several embodiments of a formulation further utilize other components and excipients. For example, sweeteners, flavors, buffering agents, and flavor enhancers to make the dosage form more palatable. Sweeteners include, but are not limited to, fructose, sucrose, glucose, maltose, mannose, galactose, lactose, sucralose, saccharin, aspartame, acesulfame K, and neotame. Common flavoring agents and flavor enhancers that may be included in the formulation of the present invention include, but are not limited to, maltol, vanillin, ethyl vanillin, menthol, citric acid, fumaric acid, ethyl maltol and tartaric acid.

Multiple embodiments of a formulation also include a surfactant. In certain embodiments, surfactants are selected from the group consisting of Tween 80, sodium lauryl sulfate, and docusate sodium.

Many embodiments of a formulation further utilize a binder. In certain embodiments, binders are selected from the group consisting of povidone (PVP) K29/32, hydroxypropylcellulose (HPC), hydroxypropylmethylcellulose (HPMC), ethylcellulose (EC), corn starch, pregelatinized starch, gelatin, and sugar.

Various embodiments of a formulation also include a lubricant. In certain embodiments, lubricants are selected from the group consisting of magnesium stearate, stearic acid, sodium stearyl fumarate, calcium stearate, hydrogenated vegetable oil, mineral oil, polyethylene glycol, polyethylene glycol 4000-6000, talc, and glyceryl behenate.

Modes of administration, in accordance with multiple embodiments, include, but are not limited to, oral, transdermal, transmucosal (e.g., sublingual, nasal, vaginal or rectal), or parenteral (e.g., subcutaneous, intramuscular, intravenous, bolus or continuous infusion). The actual amount of drug needed will depend on factors such as the size, age and severity of disease in the afflicted subject. The actual amount of drug needed will also depend on the effective concentration ranges of the various active ingredients.

A number of embodiments of formulations include those suitable for oral administration. Formulations may conveniently be presented in unit dosage form and may be prepared by any of the methods well known in the art of pharmacy. Typically, these methods include the step of bringing into association a compound of the subject invention or a pharmaceutically salt, prodrug, or solvate thereof (“active ingredient”) with the carrier which constitutes one or more accessory ingredients.

Embodiments of formulations disclosed herein suitable for oral administration may be presented as discrete units such as capsules, cachets or tablets each containing a predetermined amount of the active ingredient; as a powder or granules; as a solution or a suspension in an aqueous liquid or a nonaqueous liquid; or as an oil-in-water liquid emulsion or a water-in-oil liquid emulsion. Multiple embodiments also compartmentalize various components within a capsule, cachets, or tablets, or any other appropriate distribution technique.

Several embodiments of pharmaceutical preparations include tablets, push-fit capsules made of gelatin, as well as soft, sealed capsules made of gelatin and a plasticizer, such as glycerol or sorbitol. Tablets, in a number of embodiments, may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. Push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

In some embodiments, formulations described herein are administered in a therapeutically effective amount as part of a course of treatment of a subject having G4C2 expansion in the gene C9ORF72 (or a homolog thereof), which can cause ALS/FTD. As used in this context, to “treat” means to ameliorate at least one symptom of a disorder to be treated or to provide a beneficial physiological effect. For example, one such amelioration of a symptom could be improvement of motor function or improvement of mitochondrial function.

A therapeutically effective amount can be an amount sufficient to prevent reduce, ameliorate or eliminate the symptoms of diseases or pathological conditions susceptible to such treatment.

Dosage, toxicity and therapeutic efficacy of the compounds for clinical applications can be determined, e.g., by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to non-neoplastic cells and, thereby, reduce side effects.

Data obtained from cell culture assays or animal studies can be used in formulating a range of dosage for use in humans. If the medicament is provided systemically, the dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration or within the local environment to be treated in a range that includes the IC₅₀ (i.e., the concentration of the test compound that achieves a half-maximal inhibition of neoplastic growth) as determined in cell culture. Such information can be used to more accurately determine useful doses in humans. Levels in plasma may be measured, for example, by liquid chromatography coupled to mass spectrometry.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments. For example, several divided doses may be administered daily, one dose, or cyclic administration of the compounds to achieve the desired therapeutic result.

In various embodiments, a compound is administered at a concentration of about 100 nM to 100 mM. In various embodiments, a compound is administered at about a unit of: 100 nM, 1000 nM, 10 μM, 100 μM, 1000 μM, 10 mM, or 100 mM. In various embodiments, a compound is administered at a concentration of about 10 ng/kg to 100 mg/kg. In various embodiments, a compound is administered at about a unit of: 10 ng/kg. 100 ng/kg, 1000 ng/kg, 10 μg/kg, 100 μg/kg, 1000 μg/kg, 10 mg/kg or 100 mg/kg.

Preservatives and other additives, like antimicrobial, antioxidant, chelating agents, and inert gases, can also be present. (See generally, Remington's Pharmaceutical Sciences, cited supra.)

### Genetic Modulation

Genetic modulation can be performed on subjects by varying methodologies. In several embodiments, to increase expression, an expression cassette of the gene to be increased can be provided. Expression cassettes can be provided by transfecting into cells a plasmid, utilizing a viral vector, or integrating an expression cassette into the host genome. Integration of expression cassettes can be performed by recombination methods or the CRISPR/Cas9 system.

In many embodiments, to decrease expression, expression can be disrupted by various genetic techniques, including (but not limited to) introducing to a cell antisense oligos, introducing to a cell short-hairpin RNA, introducing into a cell a repressor, or ablating expression by disrupting the endogenous gene. Disrupting the endogenous gene can be done by recombination methods or the CRISPR/Cas9 system.

### EXEMPLARY EMBODIMENTS

The embodiments of the invention will be better understood with the several examples provided within. Provided is some exemplary data results and two attached manuscripts.

### Example 1: Treatment of Cells Expressing C9ORF72 with G4C2 Expansion

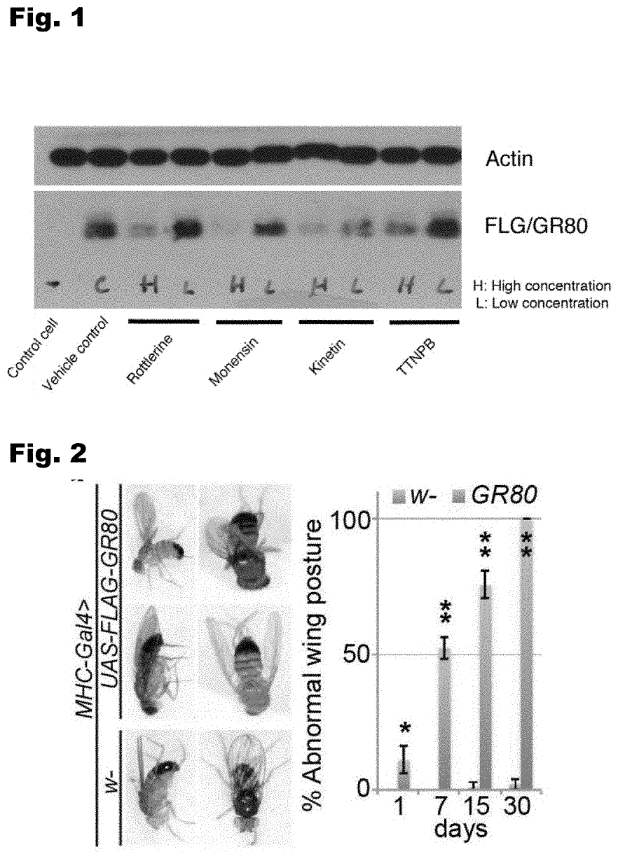

HEK23T cells were transfected with an expression cassette expressing C9ORF72 with G4C2 expansion. The cells were treated with the following compounds: Rottlerin (a mitochondrial uncoupler); Monensin (an ionophore and K+/H+ antiporter); Kinetin (an activator of PINK1, a component of the non-canonical Notch pathway); TTNPB (an activator of UCP and mitochondrial uncoupler). After treatment, the expression of levels of poly(GR) was determined. Provided in FIG. 1 is a western blot detecting protein expression levels of poly(GR) dipeptide repeats (denoted as FLG/GR80). Non-transfected HEK293T cells (control cell), and HEK293T cells transfected with Flag-GR80 plasmid and treated with vehicle (vehicle control) or the indicated drugs at low and high concentrations were subjected to immunoblot analysis for Flag-GR80 protein level. Actin serves as loading control. The drugs tested are: Rottlerin (a mitochondrial uncoupler); Monensin (an ionophore and K+/H+ antiporter); Kinetin (an activator of PINK1, a component of the non-canonical Notch pathway); TTNPB (an activator of UCP and mitochondrial uncoupler).

### Example 2: Alteration of MICOS Morphology and Mitochondrial Ion Homeostasis in C9ORF72-ALS/FTD

### Muscle Mitochondrial Defects Caused by Poly(GR) Expression in Flies

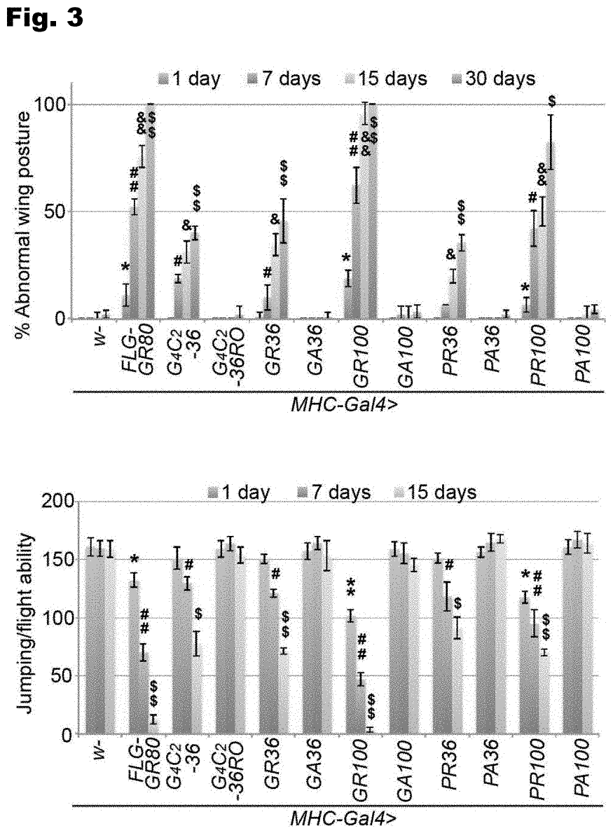

Drosophila was used as a model to assess the effect of dipeptide repeat (DPR) proteins on muscle function. Available DPR transgenic lines (GR, GA, PR, PA) were specifically expressed in fly muscle using the UAS-Gal4 system. To discern the effect of repeat length on toxicity, transgenes expressing DPR proteins of different lengths (GR36, GR80, GR100, PR36, PR80, PR100, GA36, GA80, GA100, PA36, PA100) were used. The GR80, PR80, and GA80 proteins have a Flag-tag at the N-termini to aid protein detection. A 36RO transgene expressing 36 G4C2 repeat RNA but no DPR protein was also included. The development, morphology, behavior, or survival appeared normal in all newly enclosed flies. Around 1 week after eclosion, however, about 50% of the GR100 and GR80 transgenic flies started to exhibit held-up or droopy wing postures. After 2-3 weeks, virtually 100% of the flies showed abnormal wing-posture and were flightless (FIGS. 2 & 3). GR36 transgenic flies exhibited a later-onset and less penetrant wing-posture phenotype than GR100 and GR80 flies, suggesting that repeat length correlates with toxicity (FIG. 3). Similarly, PR100 and PR36 transgenic flies also exhibited abnormal wing-postures, with PR100 flies showing a stronger phenotype than PR36 flies. PR flies exhibited conspicuously weaker phenotypes than flies expressing equal-lengthed GRs. No obvious phenotype was observed in GA or PA flies (FIG. 3). The arginine-containing DPR proteins are thus more toxic than the other DPR proteins in fly muscles, with GR seemingly more toxic than PR. The Mhc-Gal4>GR80 (GR80) flies were examined further.

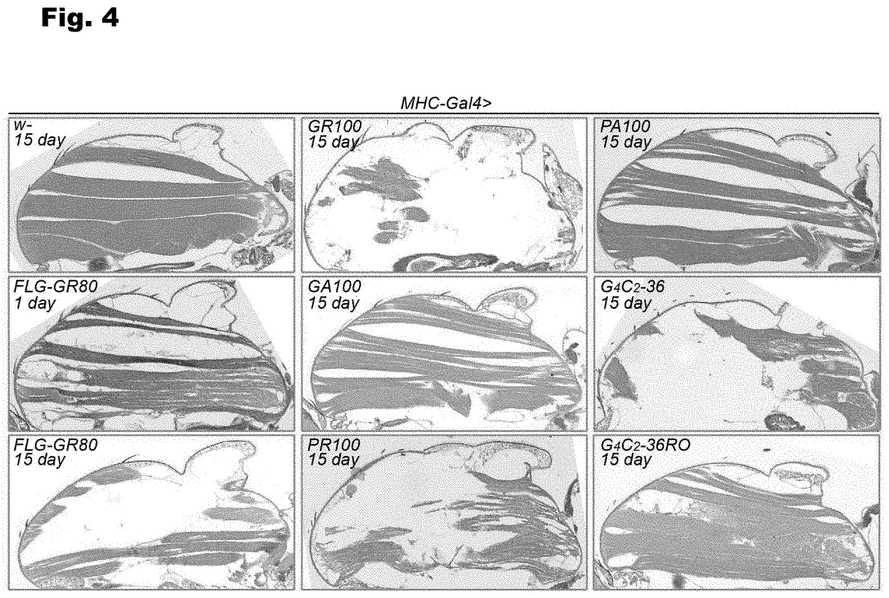

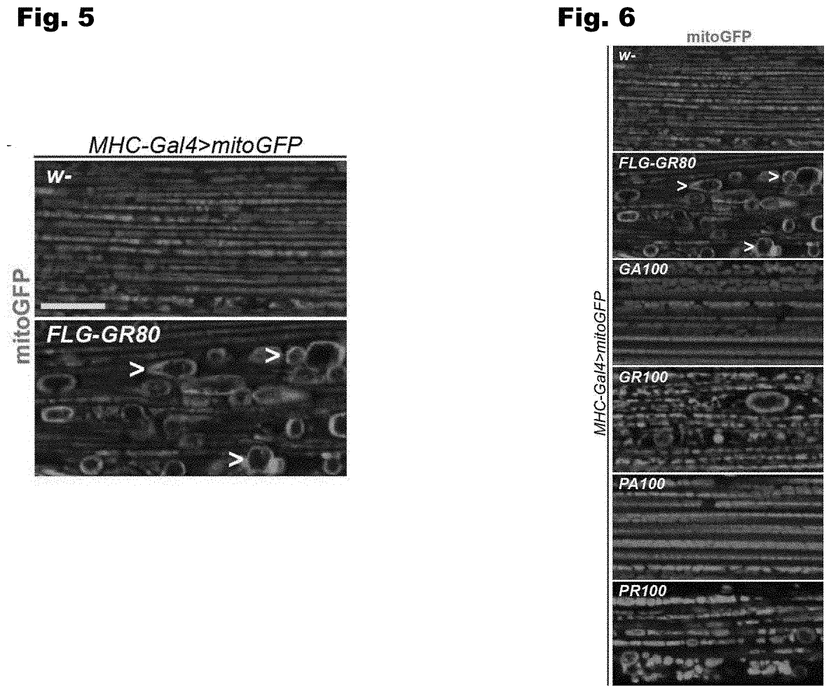

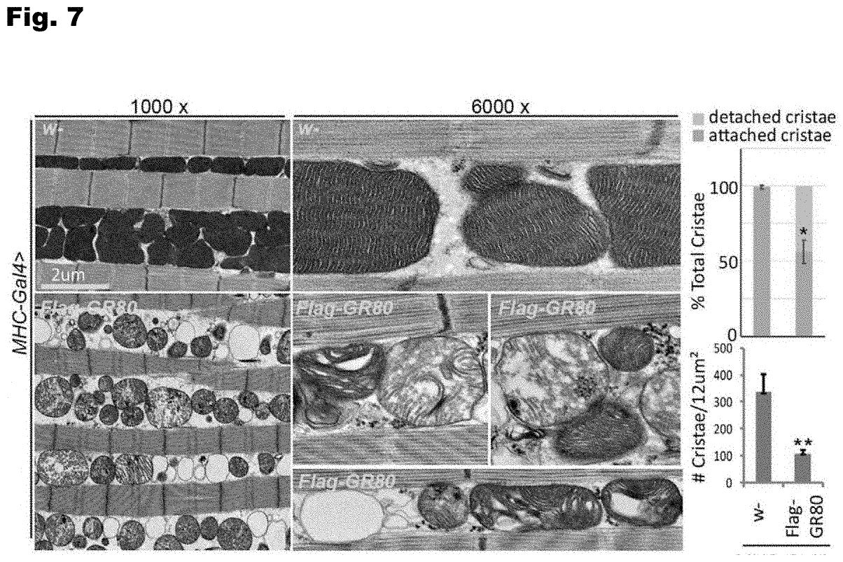

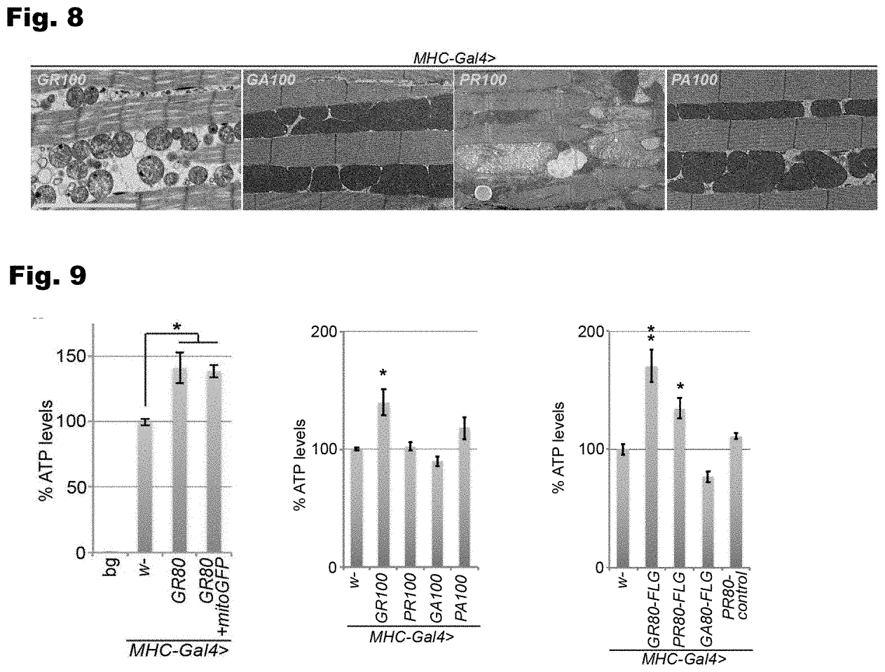

The abnormal wing-posture phenotype correlated with disruption of indirect flight muscle (IFM) integrity (FIG. 4), which is often associated with mitochondrial dysfunction. Mitochondrial morphology in GR80 fly IFM was examined using a matrix-targeted mito-GFP reporter. Compared to controls, GR80 fly muscle contained swollen mitochondria (FIG. 5). Many mitochondria were round-shaped and had vacuoles as indicated by the absence of mito-GFP internally. No mitochondrial defect was observed in IFM of GA100 or PA100 flies, consistent with their normal wing posture (FIG. 6). Transmission electron microscopy (TEM) revealed swollen mitochondria, as well as smaller-sized, round and vacuolized mitochondria devoid of electron-dense matrix material in GR80 fly muscle (FIG. 7). Closer examination further revealed aberrant cristae structures. Compared to control muscle mitochondria that have abundant lamellar cristae, each connected to the inner mitochondrial membrane via cristae junction (CJ), many mitochondria in GR80 flies were devoid of CJs, forming concentric cristae stacks inside the matrix or short tubular cristae detached from IMM, indicating that GR80 destroys CJ structures (FIG. 8). No CJ defect was observed in GA100 or PA100 muscle mitochondria.

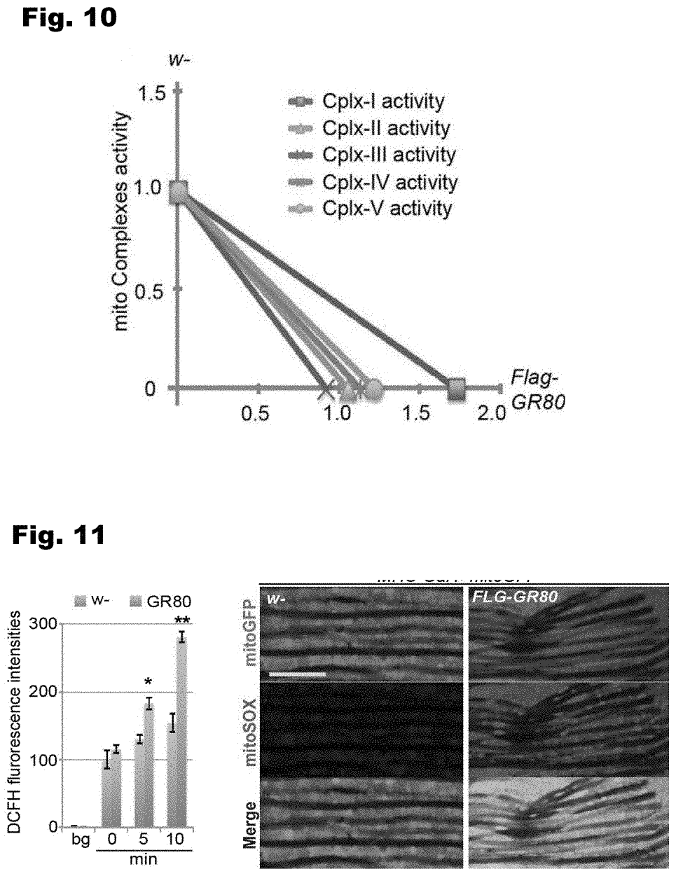

Mitochondrial function was analyzed in GR80 fly muscle. Surprisingly, increased ATP production was found in GR80 flies, whereas GA, PA and PR flies did not show significant change of ATP production (FIG. 9). This suggests that oxidative phosphorylation (OxPhos), a key function of mitochondria, was not compromised in GR80 flies; alternatively, ATP production might be compensated by some other means. To test these possibilities, respiratory activities were measured. It was found that complex I activity was elevated, whereas complex II-V were largely unaltered in GR80 flies (FIG. 10). Elevated complex-I activity might account for the increased ATP level. As complex-I is the major site of reactive oxygen species (ROS) production, it was tested whether the redox state is altered in GR80 flies. Using the ROS dyes Mito-SOX and DCFH, elevated ROS in GR80 fly IFM was detected (FIG. 11). GR80 thus causes structural and functional changes in muscle mitochondria.

### Mitochondrial Localization of Poly(GR)

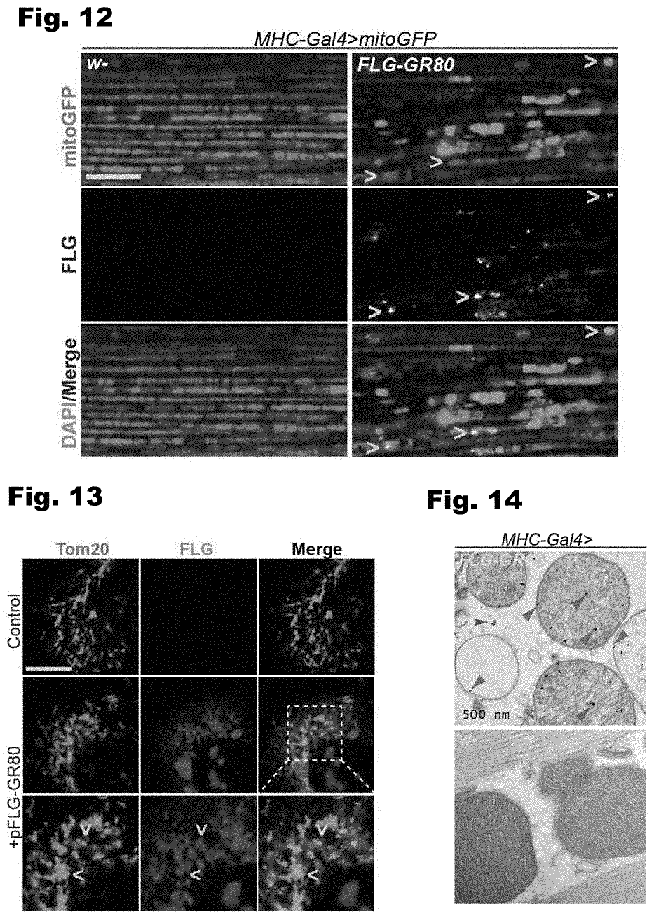

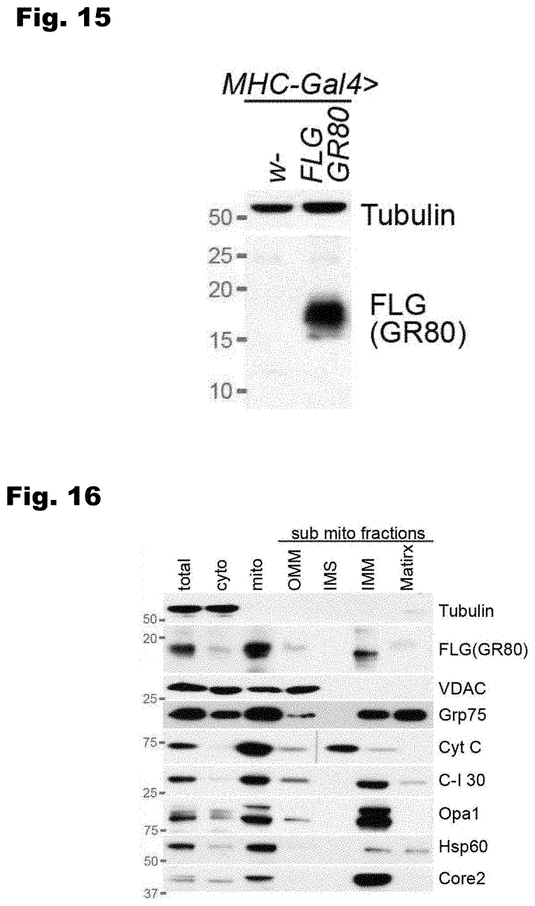

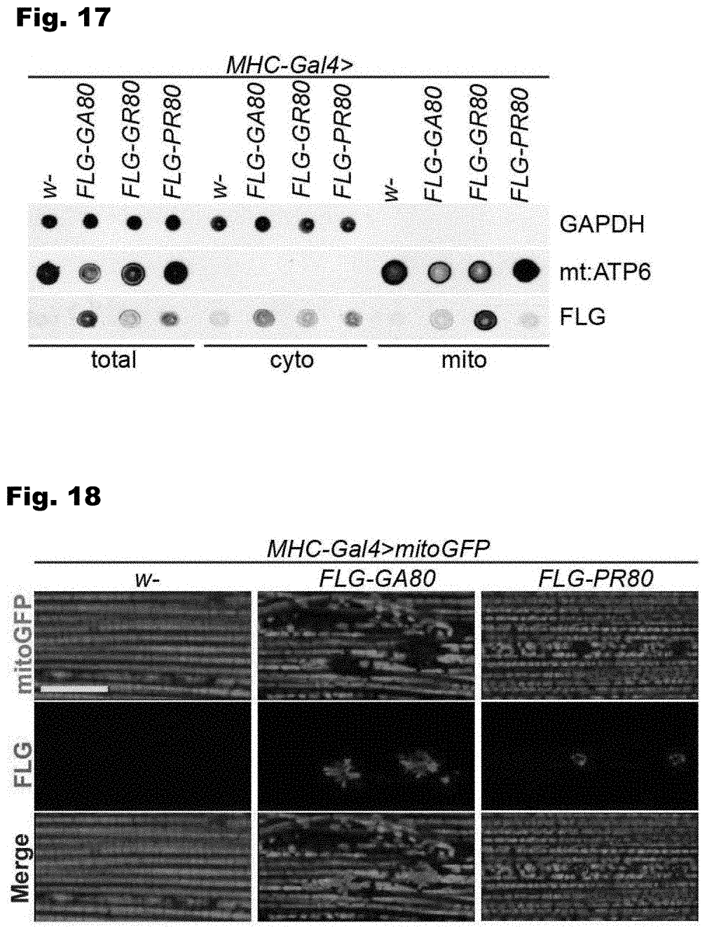

The observation of mitochondrial changes in GR80 flies raised the possibility that GR80 may associate with mitochondria. Using confocal microscopy, colocalization of GR80 with mitochondrial markers was detected in fly muscle (FIG. 12) and mammalian cells (FIG. 13), although extra-mitochondrial localization was also evident. Using immuno-EM, GR80 localization to mitochondria was confirmed (FIG. 14). To further confirm the mitochondrial localization and determine the specific compartment of GR80 localization, mitochondria was purified from GR80 fly muscle and mitochondrial subfractions were prepared. Western blot analysis indicated robust expression of GR80 in fly muscle (FIG. 15). The majority of mitochondrial GR80 was localized to IMM facing the IMS (FIG. 16), a pattern similar to the IMS/IMM marker Opa1. In contrast, GA or PR did not exhibit particular mitochondrial localization (FIGS. 17 & 18).

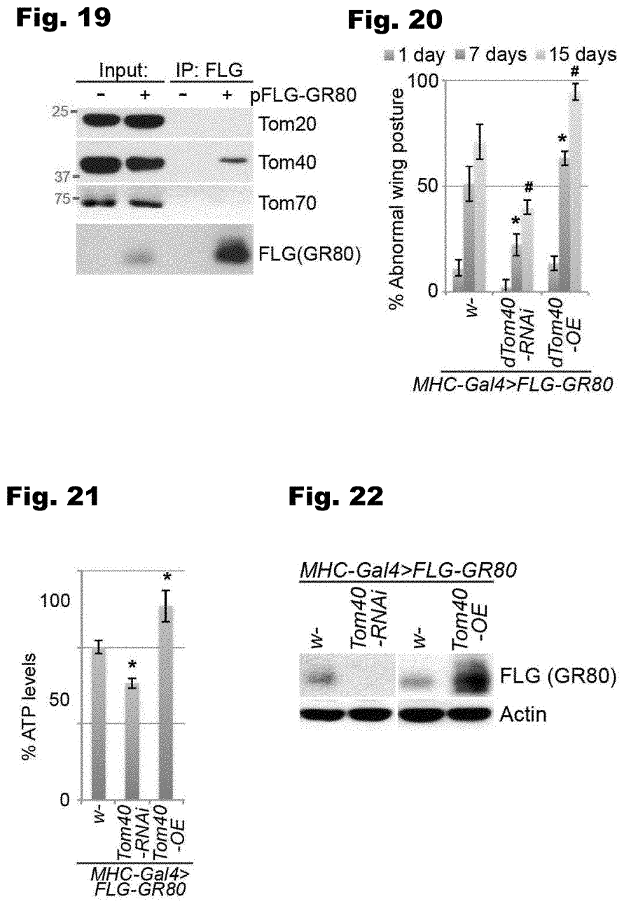

In order for GR80 to reach IMS/IMM, it most likely has to go through the TOM/TIM import complex. To test this, it was assessed whether GR80 interacts with Tom40, a key component of the TOM complex that forms the import channel. GR80 interacted with Tom40, but not Tom20 or Tom70, in co-IP assays (FIG. 19). Functionally, Tom40 overexpression (OE) increased GR80 protein level and enhanced GR80 toxicity in fly muscle, whereas partial Tom40 knockdown by RNAi had opposite effects (FIGS. 20-22).

### Opa1 Mediates the Effects of Poly(GR) on CJ

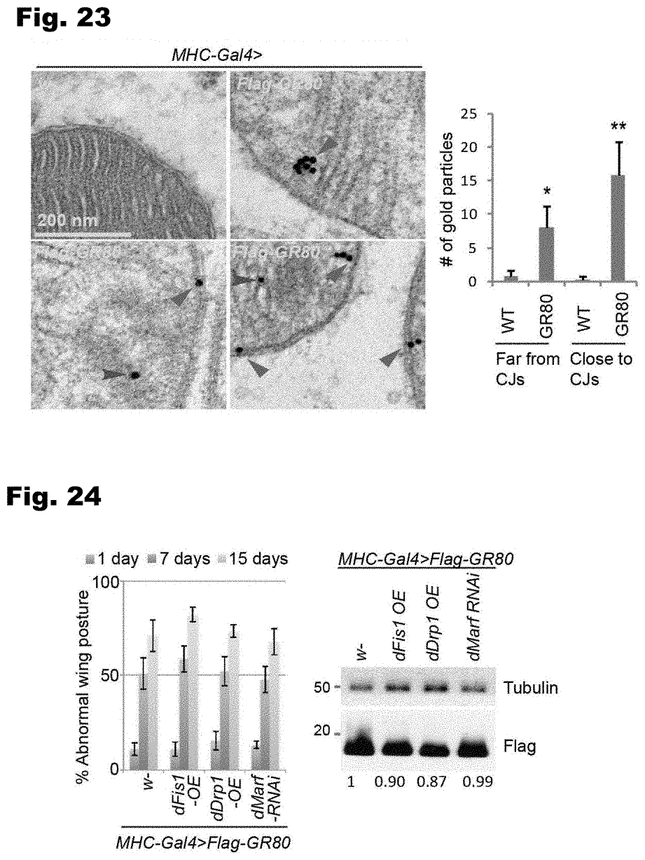

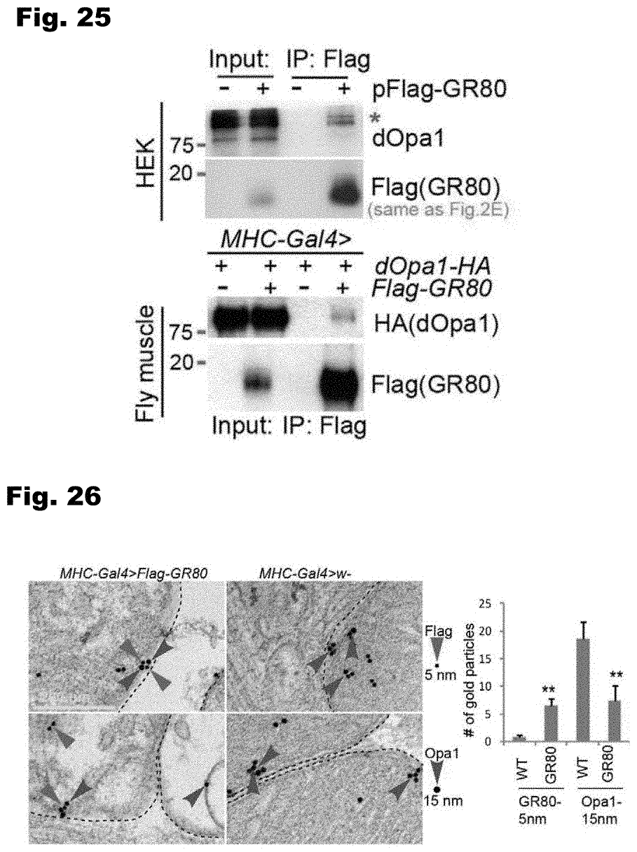

The mechanism that GR80 instigates mitochondrial toxicity was investigated. By binding to Tom40, GR80 may clog the TOM/TIM channel, thereby impairing mitochondrial import globally. Inconsistent with this idea was an observation that the import of mito-GFP was not significantly affected by GR80. Given the IMS/IMM localization of GR80 and its profound effects on CJ structure, it was tested whether GR80 may alter IMS/IMM structures related to CJ formation. Using immuno-EM, GR80 localization to CJ was observed (FIG. 23). Tuning down the expression of Opa1, a key determinant of CJ remodeling, completely suppressed GR80 effect on wing posture and cristae morphology, which was accompanied by a reduction in GR80 level (FIGS. 23 & 24). In contrast, Opa1 OE significantly enhanced GR80 toxicity and protein level (FIGS. 23 & 24). Although Opa1 is known to act in mitochondrial fusion, its modulation of GR80 toxicity is most likely unrelated to that, as altering fission-fusion balance by the loss- or gain-of-function of Drp1, Fis1, or Marf had no obvious effect on GR80 toxicity (FIG. 24). These results implicate CJ as a key site of GR80 toxicity.

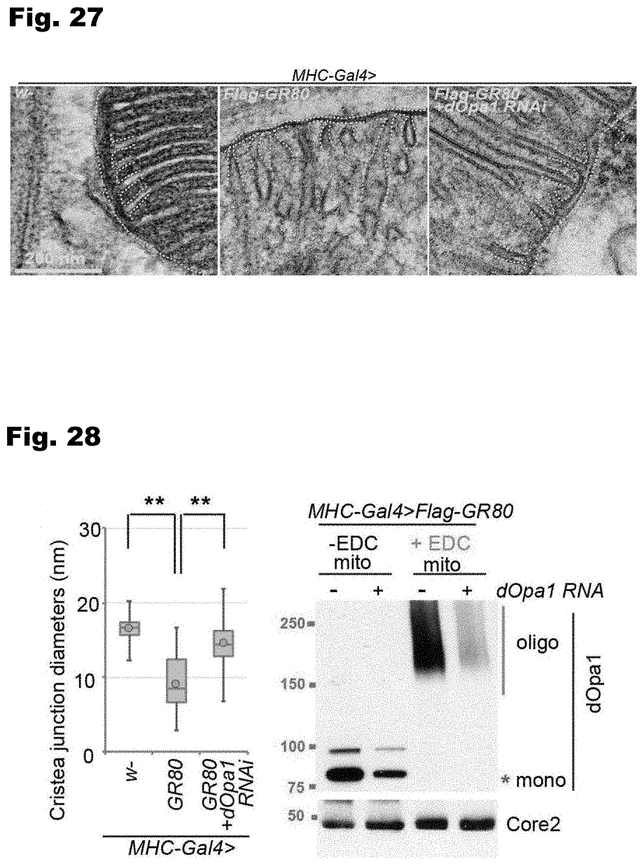

The molecular basis underlying the genetic interaction between GR80 and Opa1 was assessed. In colP assays, an interaction between GR80 and the long-form of Opa1 was detected (FIG. 25), suggesting that they may interact inside mitochondria. More supporting evidence came from immuno-EM, which showed juxtaposition of GR80 and Opa1 near CJs (FIG. 26). Next, it was tested whether GR80 may alter the formation of Opa1 oligomers, the presumed molecular staples that constrict and hold the inner membranes at CJs. It is possible that by promoting Opa1 oligomer formation, GR80 causes moderate overall stabilization of steady-state Opa1 monomers. Consistent with the biochemical data, GR80 fly muscle mitochondria exhibited narrower and presumably tightened CJs, which could be loosened by reducing Opa1 oligomer formation through Opa1 RNAi (FIGS. 27 & 28).

### Alteration of MICOS Structure and Function by Poly(GR)

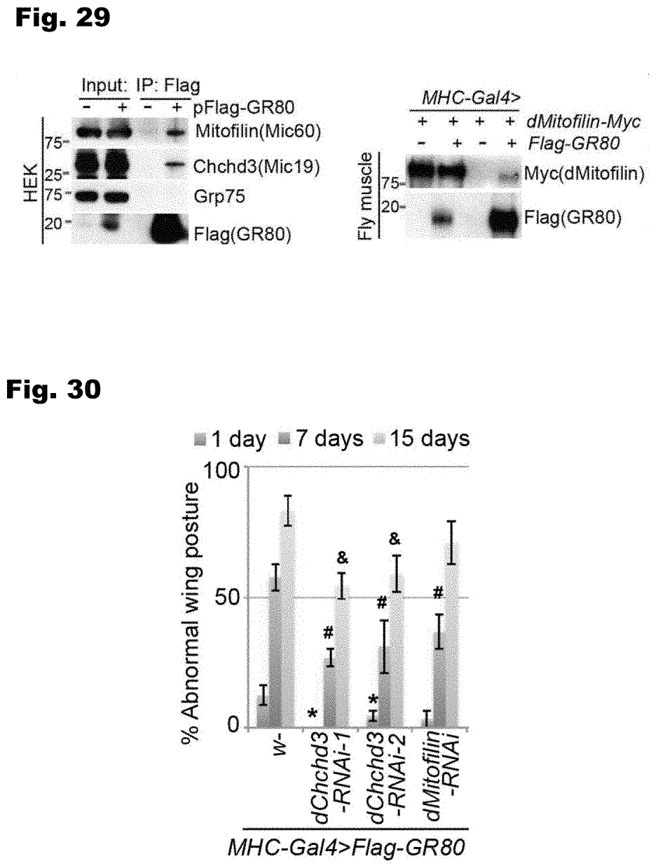

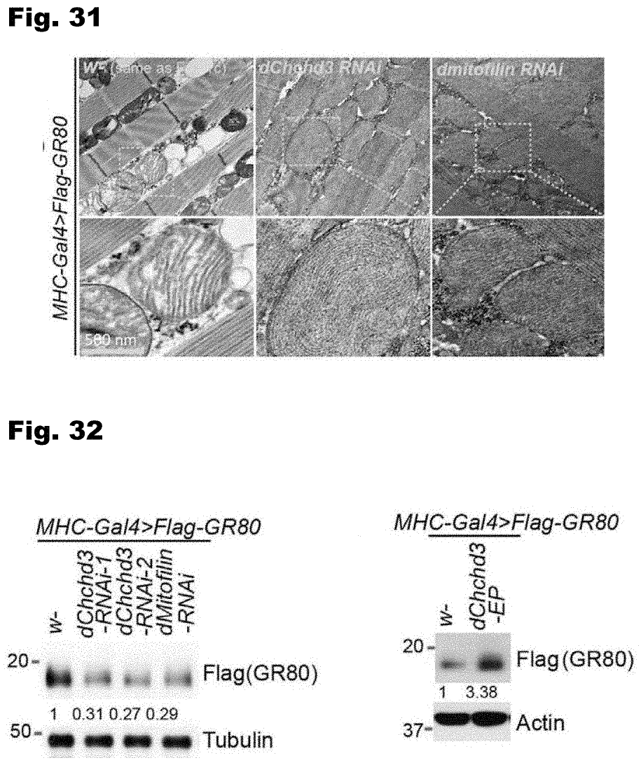

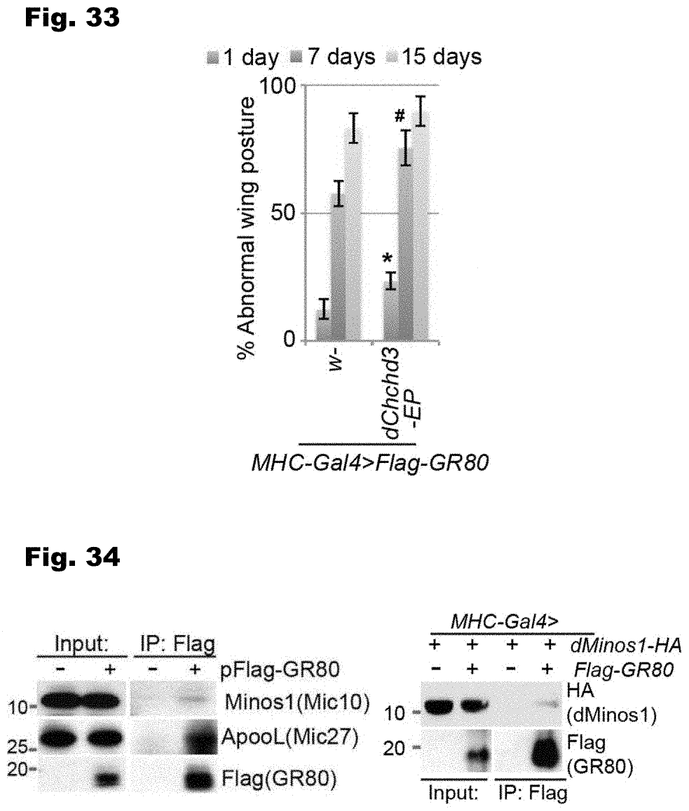

The Opa1 interactome in Drosophila includes components of the Mic60-Mic19 sub-complex of MICOS, which is centrally involved in CJ formation and function. It was further tested whether GR80 may interfere with MICOS function. In co-IP assays, GR80 exhibited physical association with MICOS components in mammalian cells (FIG. 29) and fly muscle (FIG. 29). Next, the functional consequence of these interactions was tested. As with Opa1, partial reduction of components of the Mic60-Mic19 sub-complex effectively rescued GR80-induced wing posture phenotype (FIG. 30). This was correlated with restoration of cristae structure and reduction of GR80 protein level (FIGS. 31 & 32); conversely, overexpression of Mic19 (CHCHD3) led to increased GR80 level and enhanced GR80 toxicity (FIGS. 32 & 33). It thus appears that at least part of GR80 toxicity comes from its effect on the Mic60-Mic19 sub-complex of MICOS. Moreover, by associating with MICOS components, GR80 protein became stabilized.

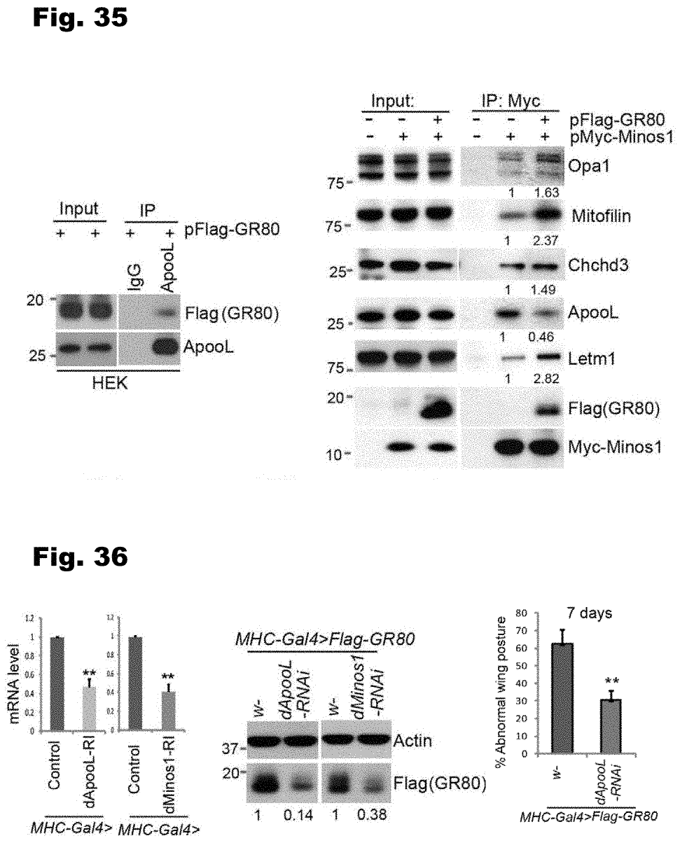

MICOS is organized into two independent sub-complexes, the Mic60-Mic19 and Mic10 (Minos1)-Mic27 (Apool) complex, which are presumably connected by Mic12 (QIL1). The relationship between GR80 and the Mic10-Mic27 complex was assessed. In co-IP assays, robust GR80 interaction with Mic27 was found, and to a lesser extent with Mic10, suggesting that Mic27 might be a more proximal subunit of MICOS interacting with GR80 (FIG. 34). Interaction was confirmed by colP in the reverse direction (IP with Mic27/Apool and detection of GR80) and under non-crosslinking conditions (FIG. 35). Mic27 is the only MICOS factor that exhibited strong colP with GR80 under such conditions, suggesting that it is the more proximal subunit of MICOS directly interacting with GR80. In the presence of GR80, the interaction between Mic10 and Mic27 was weakened, whereas the interactions of Mic10 with Mic60, Mic19, Opa1, and LETM1 were strengthened (FIG. 35), suggesting that GR80 alters MICOS dynamics and intersubunit interactions. Functionally, RNAi of Mic10 or Mic27, the efficiency of which was verified by qRTPCR (FIG. 36), reduced the GR80 protein level (FIG. 36) and rescued the GR80-induced wing posture defect (FIG. 36).

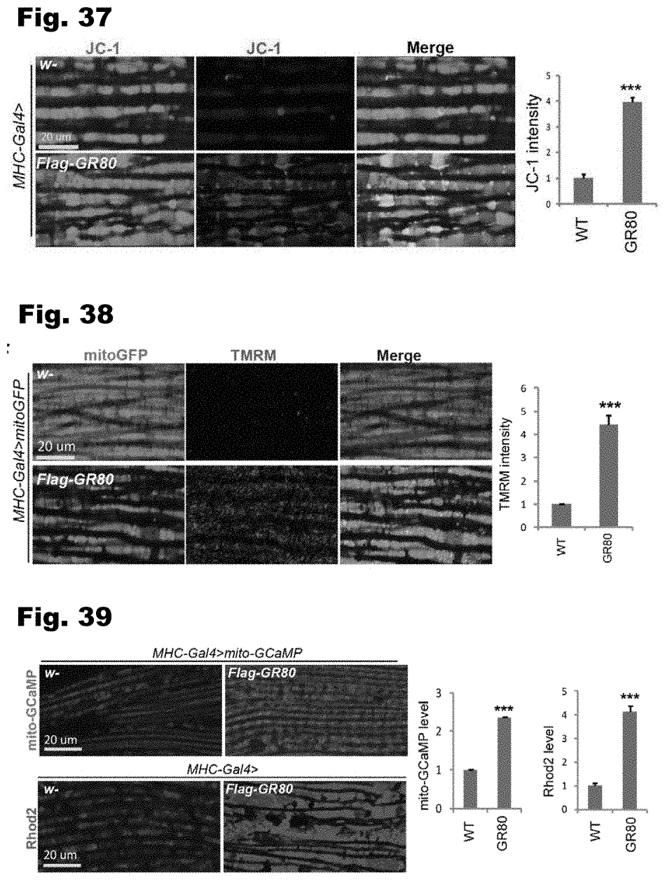

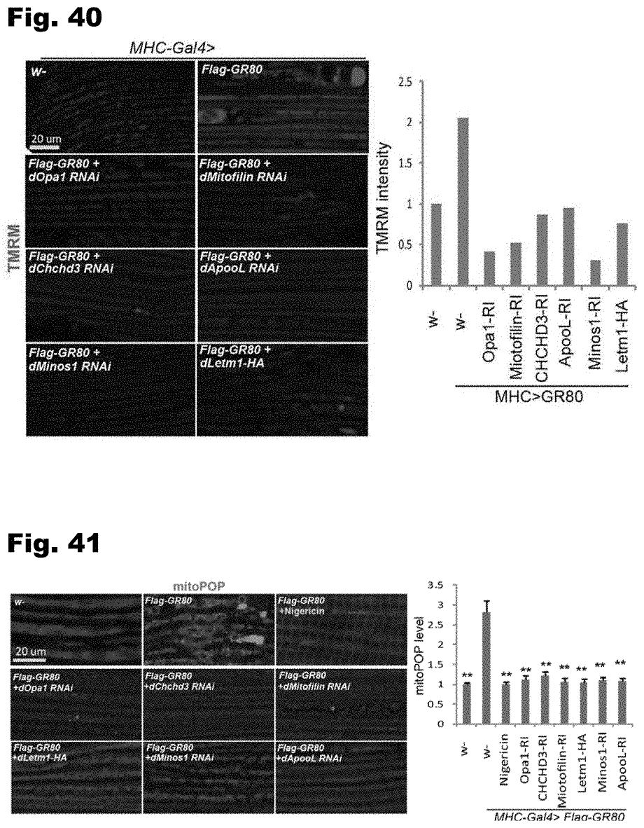

Based on the biochemical and genetic data presented, it was hypothesized that GR80 might enter MICOS and cause malformation and tightening of CJ, impairing the exchange of metabolites and osmolytes. This may impact ion homeostasis and in turn mitochondrial membrane potential (MMP). Indeed, GR80 fly mitochondria maintained a high MMP (FIGS. 37 & 38), and contained higher calcium as monitored with the genetically encoded mito-GCaMP indicator and the Rhod2 dye (FIG. 39). This is consistent with MMP being a driving force of calcium entry into mitochondria. Importantly, the high MMP in GR80 fly muscle was lowered by tuning down the expression of components of the MICOS complex (FIG. 40), suggesting that GR80 acts through MICOS to alter MMP and mitochondrial ion homeostasis. This is presumably mediated by GR80 interference with the activity of LETM1, a protein with K⁺ and Ca²⁺ transporter activity and an interacting partner of the Mic60/Mic19/Opa1 sub-complex. Indeed, there was increased mito-K⁺ level as detected with mito-POP in GR80 fly muscle (FIG. 42). This was also accompanied by a reduction of GR80 level (FIG. 42), suggesting that the restoration of mitochondrial ion homeostasis may affect the MICOS structure and, in turn, GR80 stability.

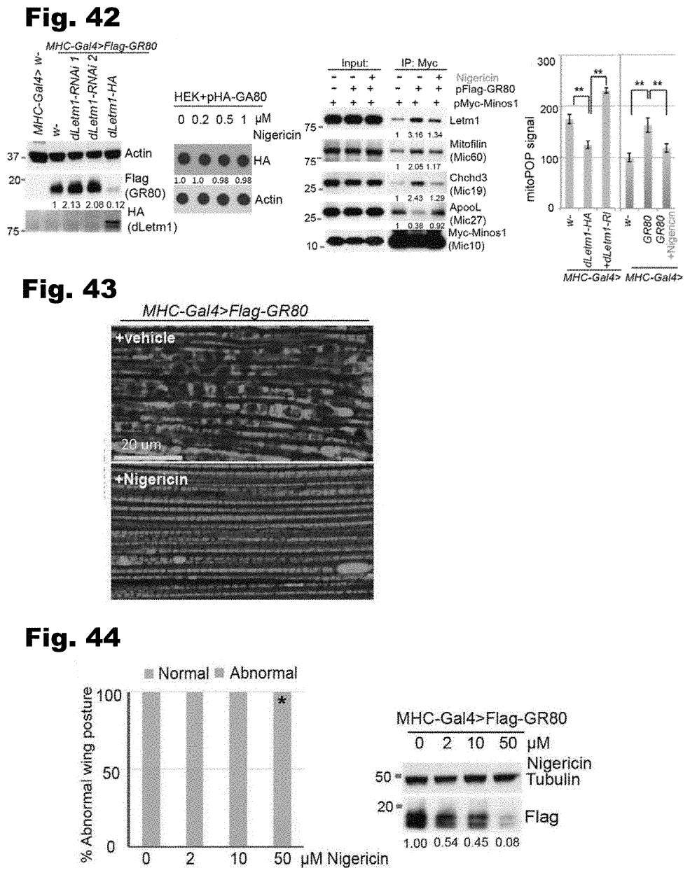

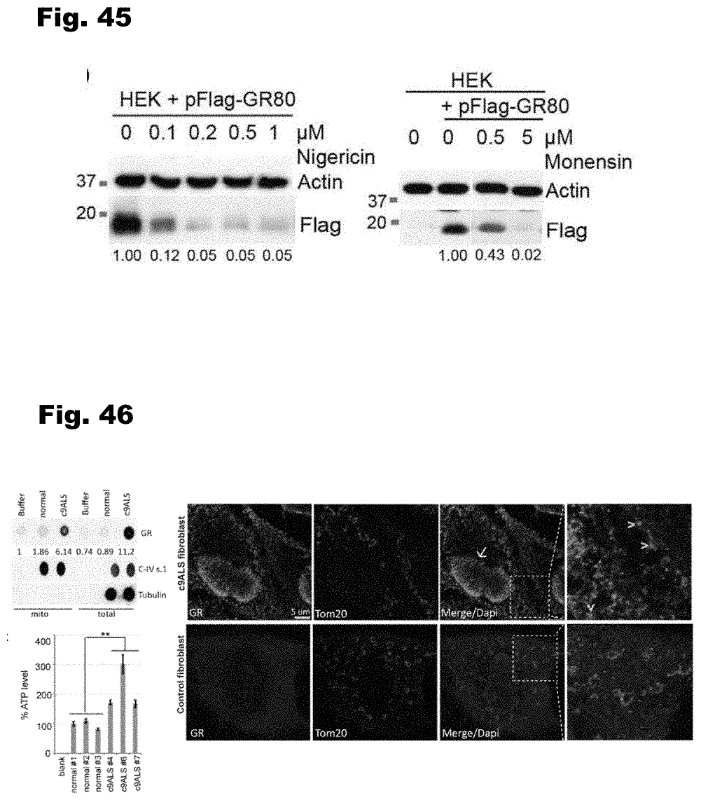

To further test whether high MMP and altered ion homeostasis contribute to GR80 toxicity, GR80 flies we treated with nigericin, a K⁺/H⁺ antiporter that can re-balance mitochondrial matrix ion levels. Treatment with low concentrations of nigericin effectively restored mitochondrial morphology (FIG. 43), accompanied by rescue of GR80 fly wing-posture defect (FIG. 44). The GR80 level in fly muscle was also significantly reduced by nigericin treatment (FIG. 44). The effect on the GR80 protein level was specific, as it did not affect the GA80 level (FIG. 42). A similar effect on GR80 was observed in mammalian cells treated with nigericin or monensin, another ionophore with similar mechanism of action (FIG. 45). Moreover, the aberrant interactions between MICOS subunits caused by GR80 (FIG. 42) and the elevated mito-K⁺ in GR80 flies were rescued after nigericin treatment (FIGS. 41 & 42). Thus, by restoring mitochondrial ion homeostasis, nigericin may affect the MICOS structure and, in turn, GR80 stability, similar to the effects of the genetic manipulation of MICOS, although other mechanisms of nigericin action cannot be excluded.

### Rescue of Mitochondrial Defects in C9-ALS/FTD Patient Fibroblasts by Genetic Manipulation of MICOS Components or by Nigericin Treatment

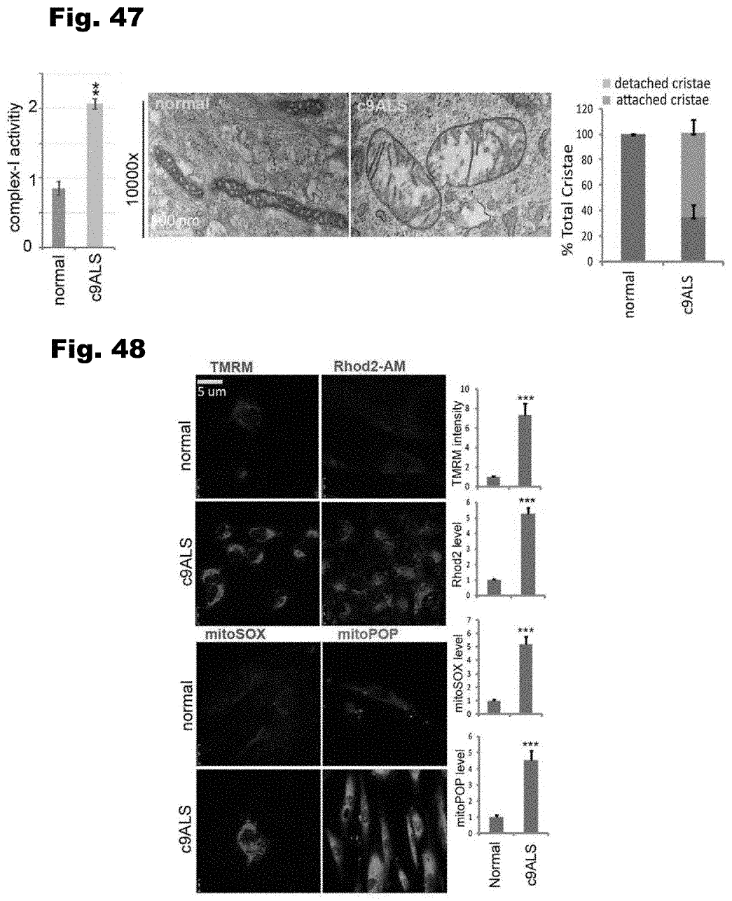

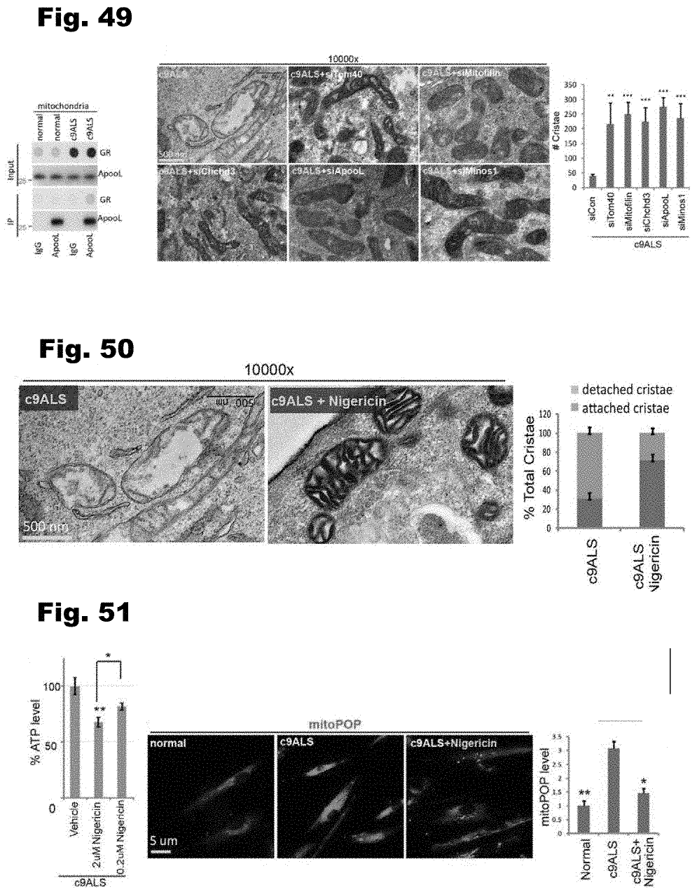

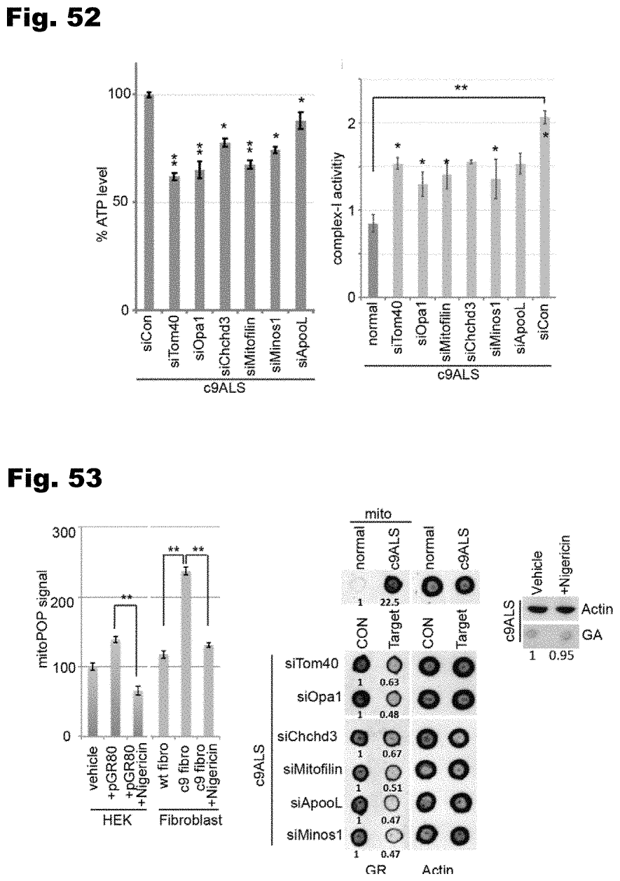

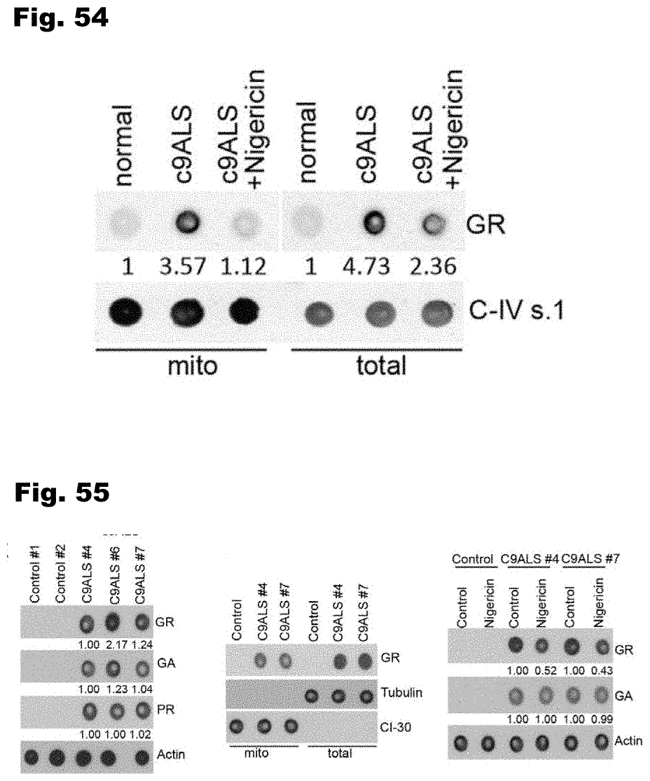

Finally, it was tested whether the pathogenic mechanisms uncovered in flies are relevant to GR toxicity in patient cells. In fibroblasts from C9-ALS/FTD patients carrying C9ORF72 G4C2 repeat expansion, GR was present in the mitochondrial fraction, as shown by dot blot (FIG. 46) and immunostaining (FIG. 46), although nuclear GR was also detected (FIG. 46). There was increased mitochondrial complex I activity (FIG. 47) and ATP production (FIG. 46) in patient cells and detachment and loss of cristae in patient mitochondria, as revealed by EM (FIG. 47). There was also increased MMP, mito-Ca²⁺, mitochondrial ROS, and mito-K⁺ in patient fibroblasts (FIG. 48). Thus, altered mitochondrial inner membrane structure and ion homeostasis are conserved features of C9-ALS. There were also GR interactions with Mic27/Apool in patient fibroblasts (FIG. 49). Nigericin treatment (FIGS. 50 & 51) or knockdown of key MICOS components (FIGS. 49 & 52) rescued the mitochondrial defects in patient fibroblasts. Nigericin treatment also reduced the GR-induced mito-K+ increase in HEK293 cells transfected with GR80 (FIG. 53). Nigericin treatment (FIG. 54) or knockdown of key MICOS components (FIG. 53) reduced the GR level in patient cells, but nigericin had no effect on the GA level (FIG. 53). The coexistence of GR and other DPRs (GA and PR), the mitochondrial accumulation of GR, and the specific effect of nigericin on the GR level were observed in independent patient fibroblast cell lines (FIG. 55). These data support the notion that poly(GR)-induced alterations of MICOS and ion homeostasis contribute to mitochondrial toxicity in C9-ALS.

### Nigericin Treatment of Drosophila

Newly hatched flies were collected and placed into vials with instant fly food (Carolina Biological Supply Company, USA) mixed with different doses (0, 2, 10 and 50 mM) of Nigericin (Cayman Chemical, 11437) from the stock solution (50 mM dissolved in DMSO). The flies were fed for 7 days and changed with fresh vial every 24 hours. After treatments, fly thoracic samples were harvested and used for sample preparation.

### RNAi in Mammalian Cells and Patient Fibroblasts

All siRNAs used for the RNAi experiments were purchased from Thermo Fisher. Briefly, HeLa cells and patient fibroblast cells were transfected with lipofectamine RNAiMAX reagent (Invitrogen, 13778) according to manufacturer's protocol. After 72 hours transfection, cells were washed with warm PBS, followed by lysis and western blot analysis. siRNA sequences used for individual are as follows: siOPA1(HSS107432), siLetm1(HSS106021), siTom40(HSS145636), siMitofilin-IMMT(HSS116992), siCHCHD3(HSS147816), siApooL(HSS175195), siMinos1(AS029RSI).

### Example 3: CAT-Tailing of ALS/FTD-Associated Poly(GR) Translationally Stalled on Mitochondrial Surface

Ribosome-associated quality control (RQC) serves to resolve stalled translation, during which untemplated Ala/Thr residues are added C-terminally to nascent chain (CAT-tailing). The role of CAT-tailing in health and disease is unclear. As described within this example, CAT-tailing of poly(GR), a dipeptide repeat derived from expanded G4C2 repeats in C9ORF72, contributes to disease. Poly(GR) is co-translationally imported into mitochondria and its translation on mitochondrial surface is frequently stalled, triggering RQC and CAT-tailing, which promotes poly(GR) stabilization, aggregation, and toxicity. Augmenting i-AAA protease YME1L efficiently cleared poly(GR), revealing mitochondria as major sites of poly(GR) production and/or storage. Moreover, non-canonical Notch signaling impinges on RQC machinery to restrain poly(GR). The conserved actions of YME1L and non-canonical Notch in animal models and patient cells support their fundamental roles in disease.

Ubiquitination and degradation of stalled or unstalled nascent peptide chains (NPCs) is widespread. RQC and CAT-tailing occur in response to stalled translation. However, the role of CAT-tailing in the context of RQC has been unclear. It can induce the heat shock response, expose lysine residues in stalled NPCs hidden in the exit tunnel, or drive degradation of stalled NPCs on and off the ribosomes. Failure in the timely removal of CAT-tailed proteins can cause proteotoxicity in yeast, and defective RQC is linked to neurodegeneration in mammals, making it imperative to elucidate the role of CAT-tailing in disease.

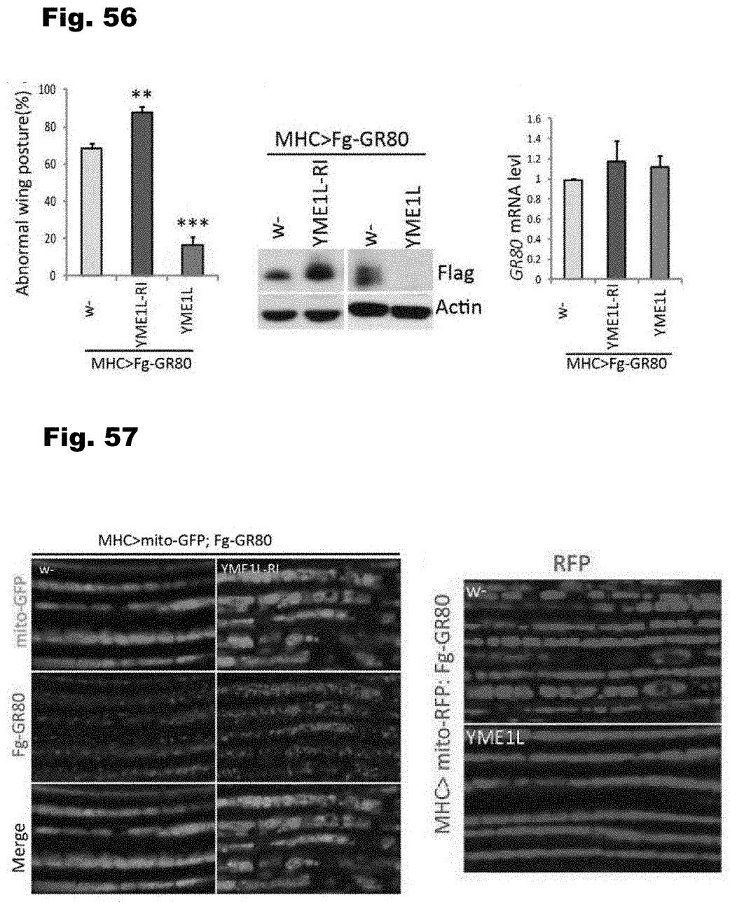

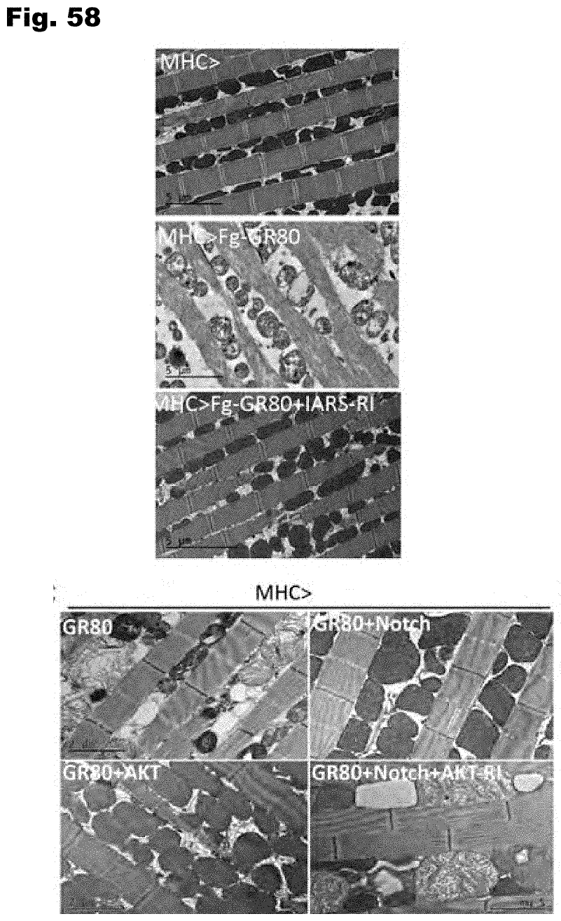

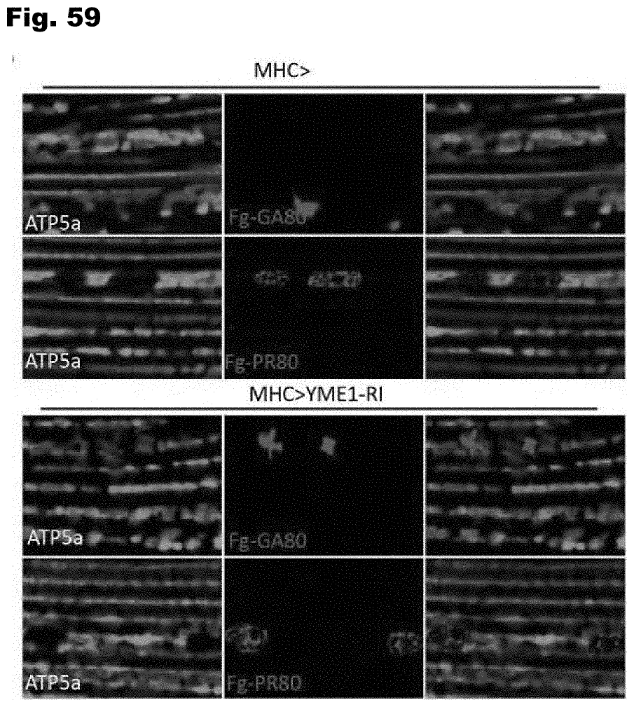

Expansion of G4C2 repeats in C9ORF72 is the most common genetic cause of ALS-FTD. Accumulating evidence point to particular pathogenicity of R-containing dipeptides translated from repeat expanded C9ORF72 mRNA. Transgenic Drosophila expressing N-terminally Flag-tagged GR₈₀ repeats (Flag-GR80) were used to elucidate the pathogenic mechanism of poly(GR). When expressed in the muscle under Mhc-Gal4 control, Flag-GR80 caused prominent toxicity (held up or droopy wing posture and flightless), resembling the phenotype caused by mitochondrial dysfunction and flight muscle degeneration seen in PINK1 mutant flies. Consistent with a mitochondrial toxicity of GR80, in a genetic modifier screen the mitochondrial i-AAA protease YME1L, a major regulator of mitochondrial protein quality control, was identified as a strong modifier of GR80 toxicity. Overexpression of YME1L (YME1L-OE) rescued GR80-induced wing posture phenotype, whereas YME1L RNAi had opposite effect (FIG. 56). Importantly, YME1L-OE dramatically reduced, whereas YME1L RNAi increased Flag-GR80 protein level (FIG. 56), whereas GR80 mRNA level was not affected (FIG. 56), consistent with YME1L regulating poly(GR) at the protein level. Moreover, GR80 formed more prominent aggregates colocalizing with mitochondria upon YME1L RNAi (FIG. 57). Examination of muscle mitochondria using the mito-GFP reporter (FIG. 57) or by EM (FIG. 58) showed that Flag-GR80 caused severe defects (swollen or small round-shaped mitochondria with marked vacuolization), which were rescued by YME1L-OE (FIG. 57). YME1L did not affect the level of poly(PR) or poly(GA), which were localized primarily to extra-mitochondrial sites (FIG. 59).

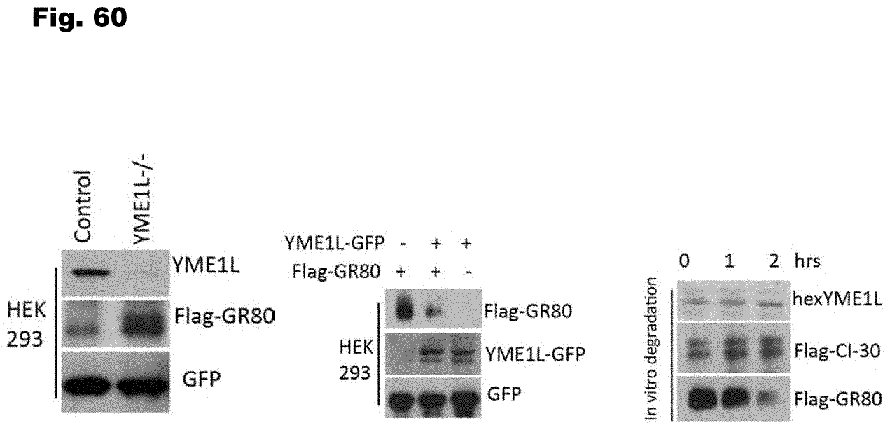

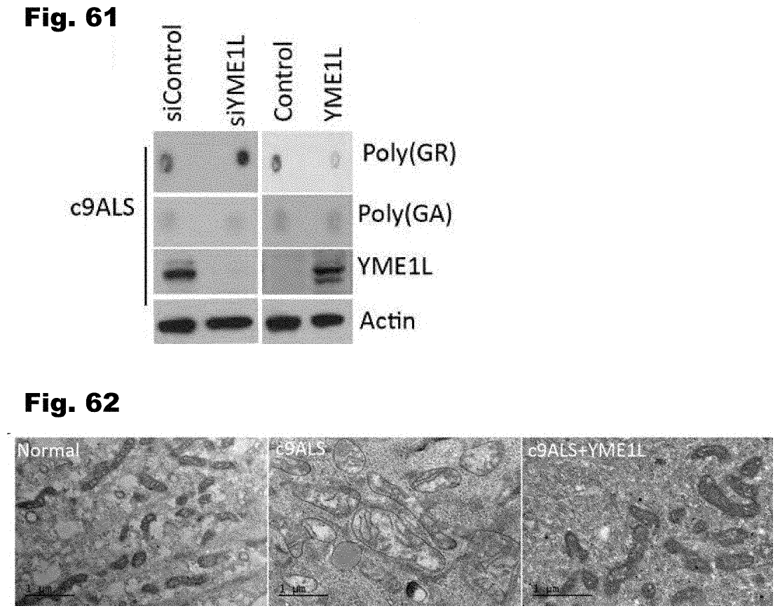

Flag-GR80 accumulated to higher levels in YME1L knockout HEK293 cells generated by CRISPR-Cas9 (FIG. 60), whereas GFP-tagged YME1L dramatically reduced GR80 level (FIG. 60). In patient fibroblasts containing expanded G4C2 repeats, poly(GR) but not poly(GA) level was reduced by YME1L cDNA and elevated by YME1L siRNA transfections (FIG. 61). Moreover, YME1L-OE rescued mitochondrial defects (swelling, vacuolization, and loss of cristae) seen in patient cells (FIG. 62). These results implicate mitochondria as major sites of poly(GR) toxicity and YME1L a key regulator of poly(GR) metabolism. Using an in vitro system that recapitulates the ATP-dependent protease activity of YME1, it was found that Flag-GR80 could be efficiently degraded by YME1L. In contrast, YME1L failed to degrade a similarly prepared mitochondrial protein (Flag-C-130), demonstrating the substrate specificity of YME1L toward GR80 (FIG. 60). These results strongly implicate YME1L, a key mitochondrial protease, in protecting against the build-up and toxicity of poly(GR).

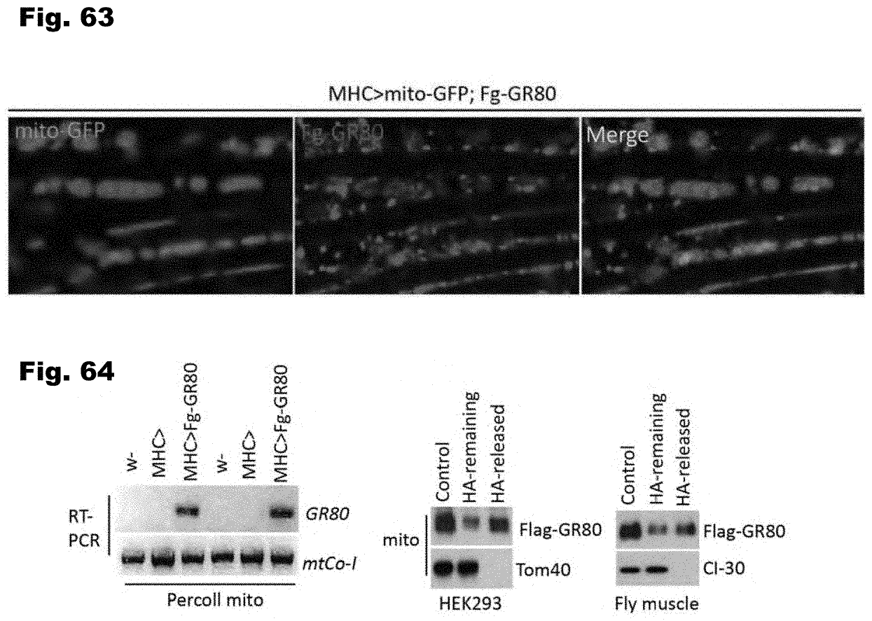

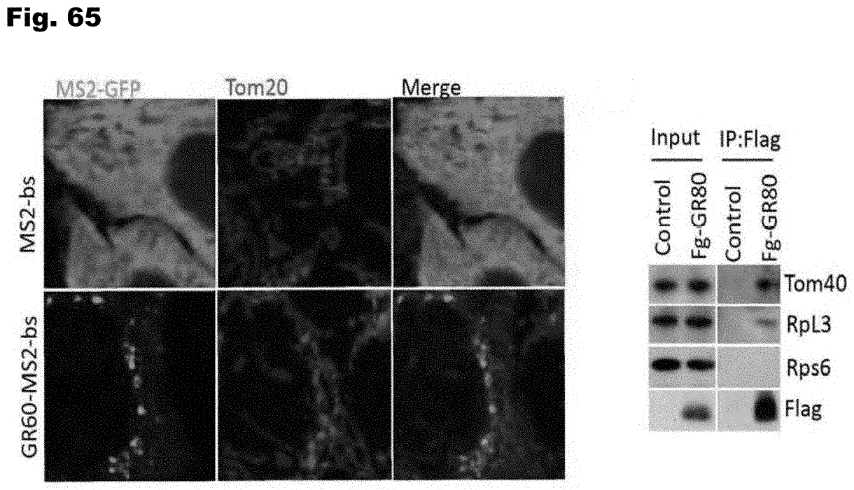

The translocation of poly(GR) into mitochondria to cause toxicity was investigated. GR80 may be synthesized in the cytosol and imported into mitochondria through the TOM complex. Import of nuclear-encoded mitochondrial proteins often involves a targeting sequence (MTS) rich in charged amino acids. MTS mimicry by the positively charged GR repeats may engage the TOM complex and lead to GR80 import into mitochondria. The TOM complex can also interact with MTS newly emerging from the exit tunnel, leading to recruitment of NPC and associated mRNP/ribosome to mitochondria outer membrane (MOM). It was found that not only GR80 protein showed mitochondrial localization (FIG. 63), GR80 mRNA was also present on percoll gradient-purified mitochondria (FIG. 64), consistent with GR80 engaging in co-translational import. To further test this idea, mitochondria was purified from HEK293 cells or fly muscle expressing Flag-GR80, and then treated with hydroxylamine (HA), which releases NPC from translating ribosomes. Flag-GR80 was efficiently released from mitochondria by HA, supporting that it is translated on MOM (FIG. 64). An MS2-binding site (bs)-tagged GR60 mRNA was generated and tagged with GFP to track the localization of the mRNA. This revealed mitochondrial localization of GR60 mRNA (FIG. 65). These results, together with GR80 interaction with TOM40 (FIG. 65), the constituent of the import channel, support that GR80 is co-translationally imported.

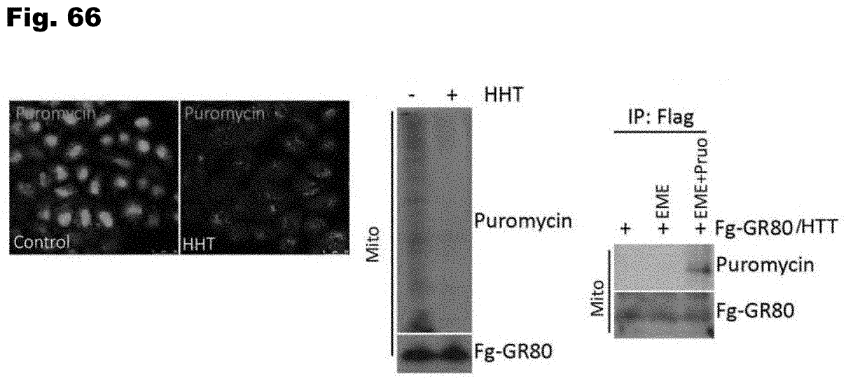

Given that GR80 is positively charged, and that electrostatic interaction between positively charged AAs in NPCs and negatively charged ribosome exit tunnel can cause ribosome stalling, it was hypothesized that the translation of GR80 mRNA on MOM is frequently stalled, possibly explaining the difficulty in detecting long poly(GR) from patient cells or tissues by standard western blotting. To test this, cells were treated with puromycin, which can be incorporated into elongating as well as stalled NPCs. Pre-treatment of cells with homoharringtonine (HTT) to inhibit new translation while allowing active/elongating ribosomes to run off, would result in puromycin labeling of mainly stalled NPCs. Intriguingly, while global translation was sensitive to HTT, mitochondria-associated GR80 was not (FIG. 66), and nascent Flag-GR80 on MOM was ribosome-stalled (FIG. 66).

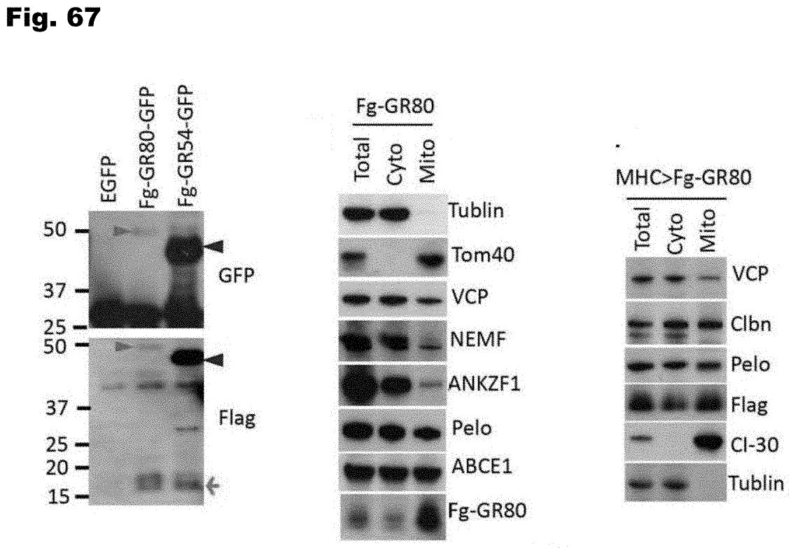

To further assess stalled translation of GR80, HEK293 cells were transfected with a Flag-GR80-GFP construct. Flag-positive but GFP-negative short peptides were detected, suggesting that these peptides were presumably arrested translation products, and a very small amount of full-length Flag-GR80-GFP. In contrast, HEK293 cells expressed the full-length protein of Flag-GR54-GFP construct at higher levels, despite also producing arrested translation products, suggesting that poly(GR) causes ribosome stalling and longer repeats cause stronger stalling (FIG. 67).

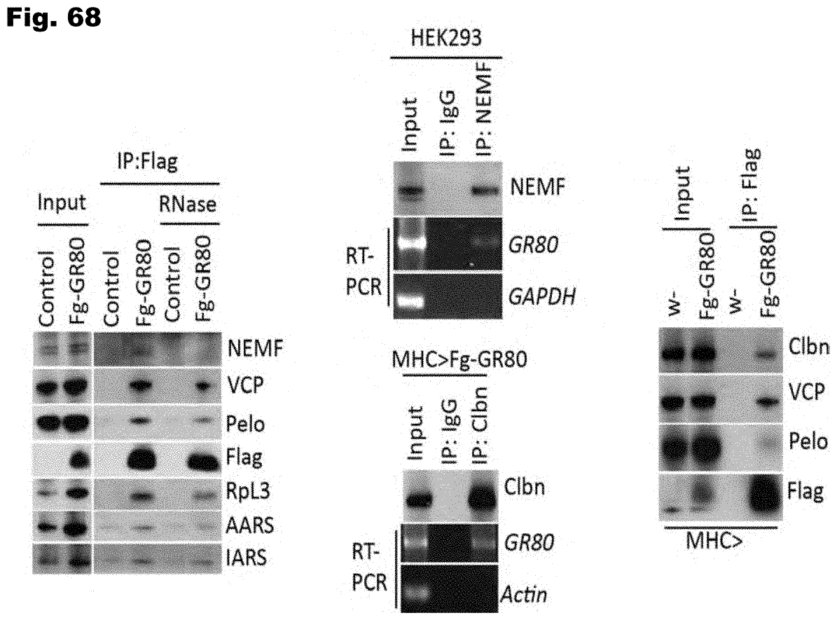

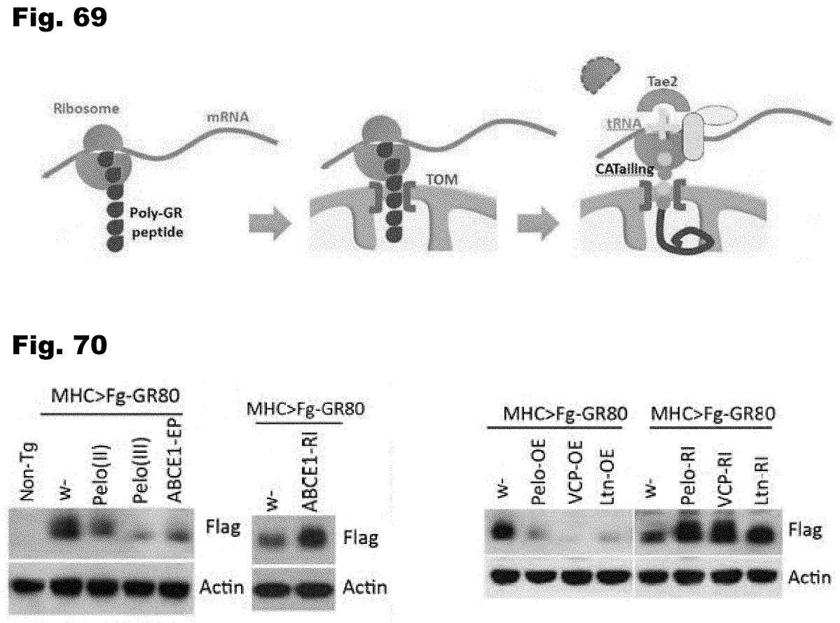

Ribosome stalling activates co-translational quality control mechanisms that target aberrant or incompletely synthesized/folded NPCs for degradation. The first step toward resolving stalled ribosome is separation of 40S and 60S subunits, with the NPCs still present in a peptidyl-tRNA/60S complex. RQC factors were detected in the mitochondrial fraction of GR80-expressing HEK293 cells (FIG. 67) or fly muscle (FIG. 67). In the mitochondrial fraction, GR80 preferentially associated with 60S (FIG. 65), and exhibited RNA-dependent association with RQC factors (FIG. 68). Moreover, direct binding of RQC factor Clbn/NEMF to GR80 mRNA was detected by CLIP assays (FIG. 68). These results, together with the association of GR80 with Tom40 and 60S, depicted a picture of active quality control of ribosome-stalled poly(GR) anchored to MOM by TOM (FIG. S2G).

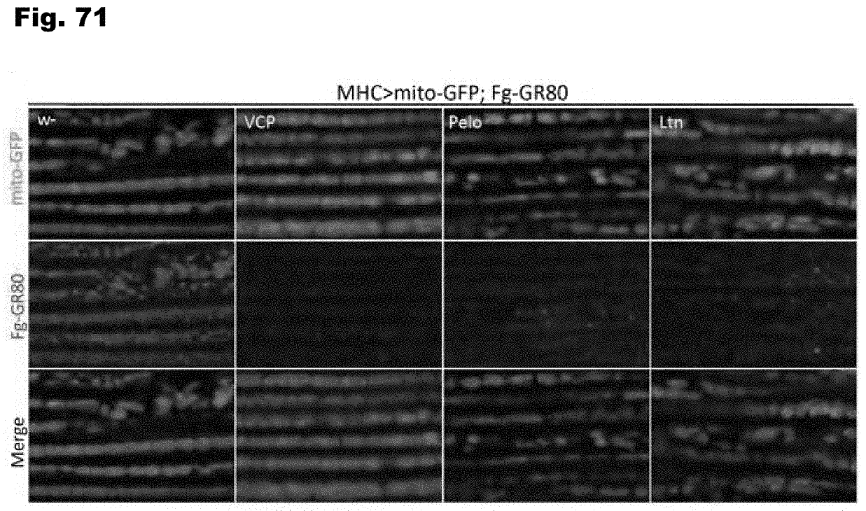

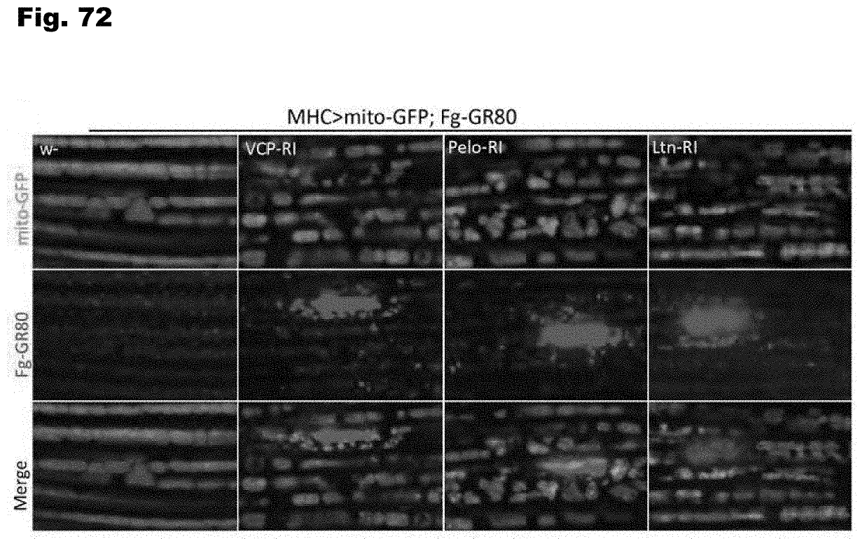

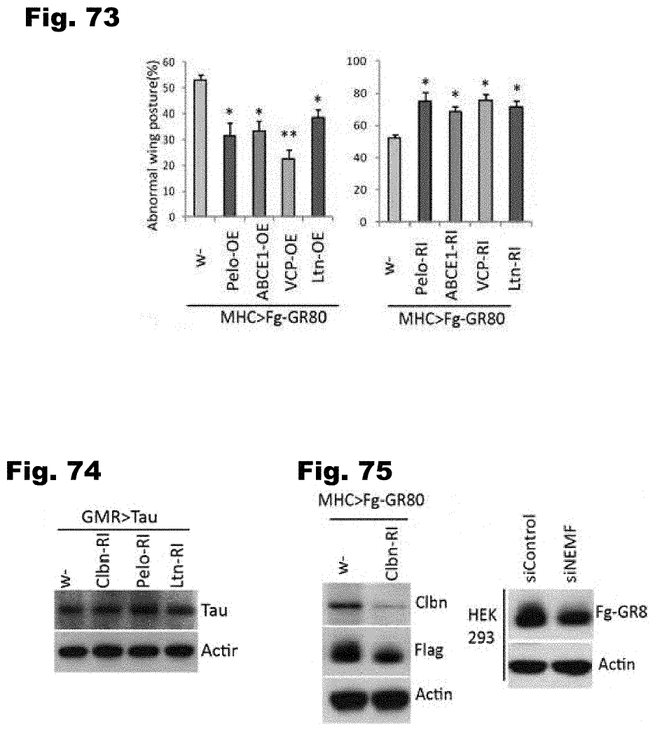

Because GR80 also associated with RQC factors in fly muscle (FIG. 68), the in vivo effect of co-translational quality control was examined. Overexpression of Pelo and ABCE1, ribosome-splitting factors needed for RQC, resulted in dramatic reduction of GR80 level (FIG. 70). Overexpression of fly homologues of core components of RQC (Ltn1, VCP) also dramatically reduced GR80 level (FIGS. 70 & 71). Conversely, loss-of-function of Ltn1, VCP1, or Pelo led to increased GR80 (FIG. 70) and aggregate formation in the cytosol or in association with mitochondria (FIG. 72). Correlating with effects on GR80 level, overexpression of the RQC factors rescued GR80-induced wing-posture defect, whereas their loss-of-function worsened that (FIG. 73). These results strongly support the notion that the RQC pathway is normally involved in restraining the expression of poly(GR). To test the specificity of the RQC effect, FTD-related tau was examined. Its level was not affected by the genetic manipulations of RQC factors (FIG. 74), suggesting that the RQC pathway preferentially affects GR80.

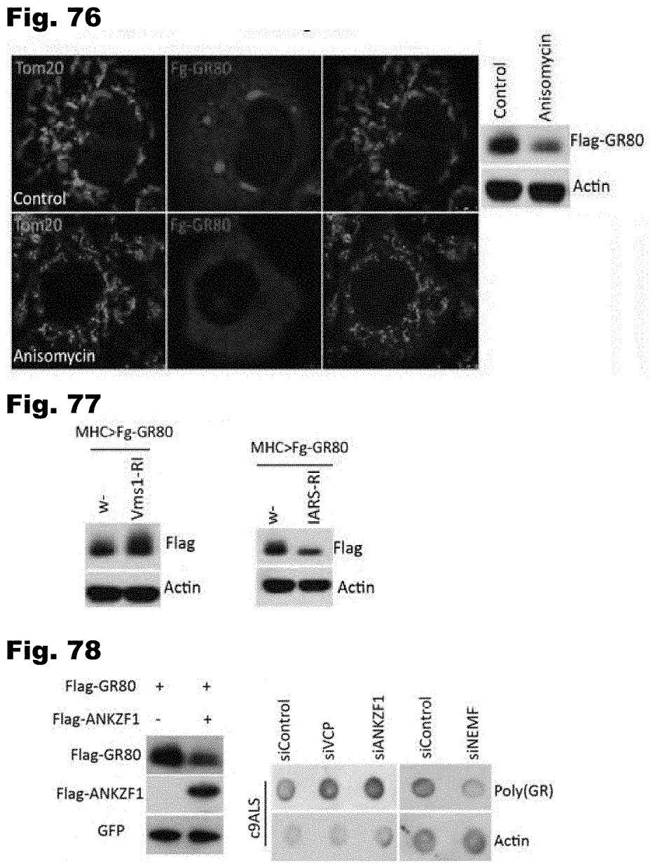

Ribosome-stalled NPC is modified by a non-templated, 40S-independent CAT-tailing process that depends on Tae2/Clbn/NEMF family proteins. CAT-tailed substrates escaping the quality control system can accumulate and form aggregates. Unlike the other RQC factors whose RNAi resulted in accumulation of GR80 (FIG. 74), Clbn or NEMF RNAi reduced GR80 level (FIG. 75). Anisomycin, which can inhibit CAT-tailing in vitro and in vivo, had similar effect (FIG. 76). Clbn/NEMF RNAi led to not only reduced GR80 level but also removal of some slightly higher MW signals (FIG. 75), presumably reflecting the elimination of CAT-tails. Consistently, RNAi of a fly homolog of Vms1, a stress-responsive mitochondrial RQC factor that antagonizes CAT-tailing by cleaving peptidyl-tRNA bond, increased high MW GR80 signal and total GR80 abundance (FIG. 77), whereas overexpression of mammalian Vms1 (ANKZF1) had opposite effect (FIG. 78). In C9ALS fibroblasts, ANKZF1 RNAi increased, whereas NEMF RNAi decreased poly(GR) (FIG. 78). Thus steady-state poly(GR) level is positively correlated with a CAT-tailing-like process.

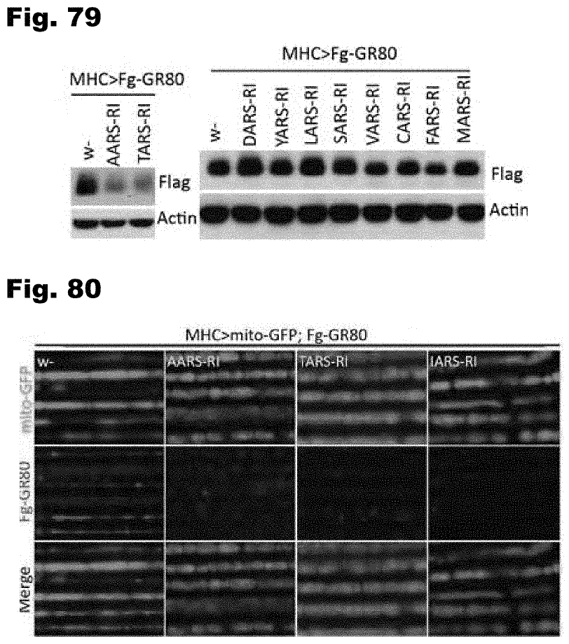

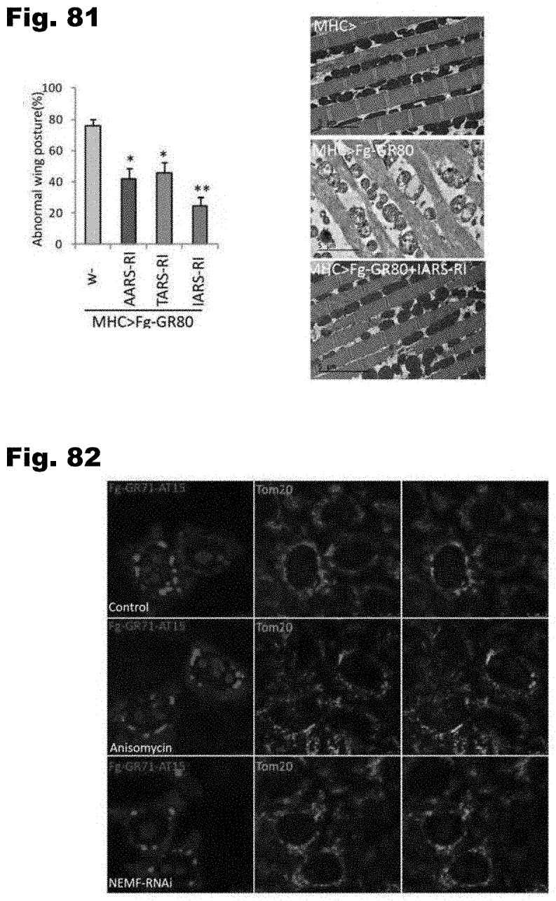

The role of Ala- or Thr-tRNA synthetase (AARS or TARS) was examined. AARS and TARS RNAi preferentially reduced GR80 level, especially the higher MW signal (FIG. 79), its aggregation (FIG. 80), and muscle toxicity (FIG. 81). Inhibiting IARS had similar effect (FIGS. 80 & 81). The specificity of the ARS effect was shown by the lack of effect of the other ARSs tested (FIG. 79). Thus AARS/TARS, and possibly select other ARSs (e.g., IARS), appear to be involved in GR80 CAT-tailing, and that CAT-tailing confers stabilization, aggregation, and toxicity. Further supporting this notion, poly(GR) containing an artificial CAT-tail (GR71-AT15) formed prominent aggregates on mitochondria on its own, and its level was not affected by NEMF RNAi or anisomycin treatment (FIG. 82).

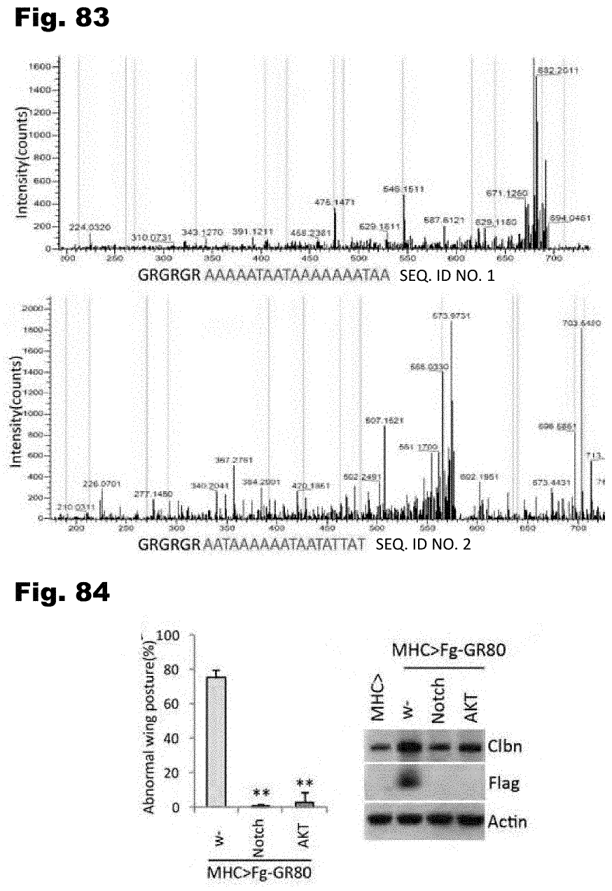

To further test the CAT-tailing hypothesis, Mass Spec was performed on Flag-GR80 purified from Mhc-Gal4>Flag-GR80 flies by denaturing IP. Based on in vivo ARS requirements, it was assumed that A/T/I might be the preferred AAs added to the C-terminus of GR repeats, and custom libraries were built with variations of A/T/I-containing C-terminal extensions. Search of tandem mass spectra against the databases identified GR peptides containing A/T/I, and peptide spectra matching collision-induced fragmentation (FIG. 83).

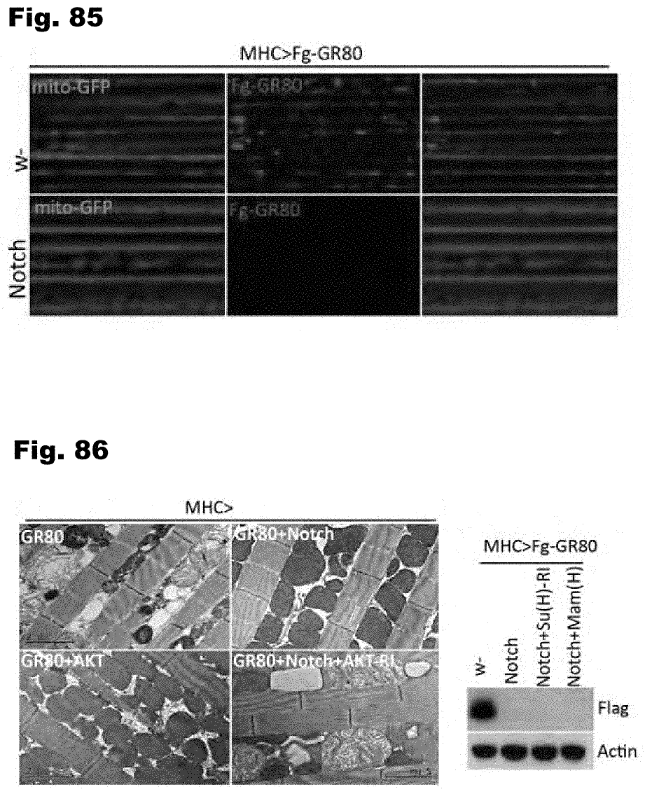

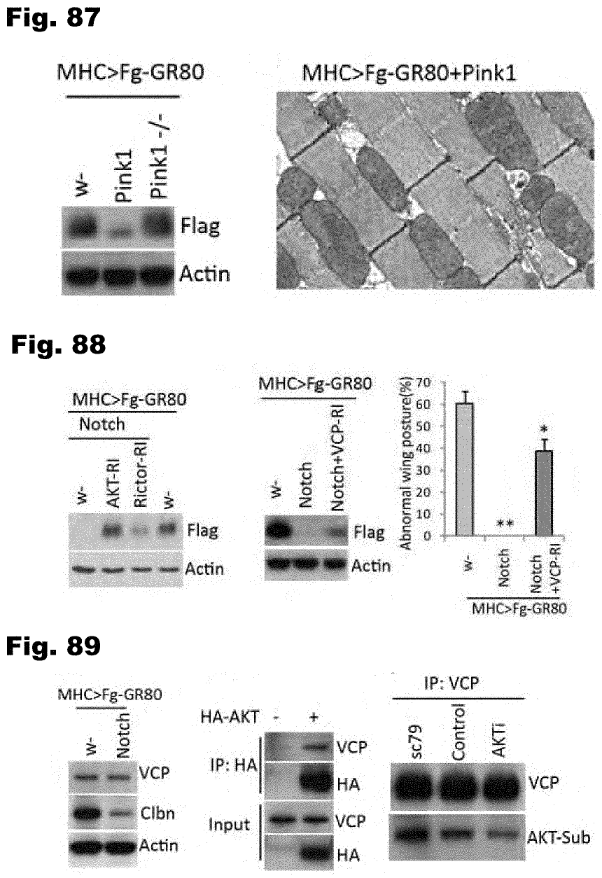

Genetic modifiers related to the regulation of poly(GR) was analyzed. The strongest modifier identified was Notch, whose overexpression resulted in the most complete suppression of GR80 toxicity (FIG. 84). This was correlated with diminished GR80 level (FIGS. 84 and 85), and full restoration of mitochondrial morphology (FIG. 86). Although Notch is best known to act through nuclear factors such as mastermind and Suppressor of Hairless [Su(H)] to regulate transcription by canonical Notch signaling, Su(H) and Mam were not required for the inhibition of GR80 by Notch (FIG. 87). In contrast, PINK1, mTORC2 and its substrate AKT, key components of a mitochondria-associated non-canonical Notch pathway, exhibited similar effect as Notch in restraining GR80 (FIGS. 84, 86 & 87). Consistent with these genes operating in a common pathway, the effect of Notch in inhibiting GR80 was abolished by knocking down mTORC2 component Rictor, or AKT (FIGS. 86 & 88).

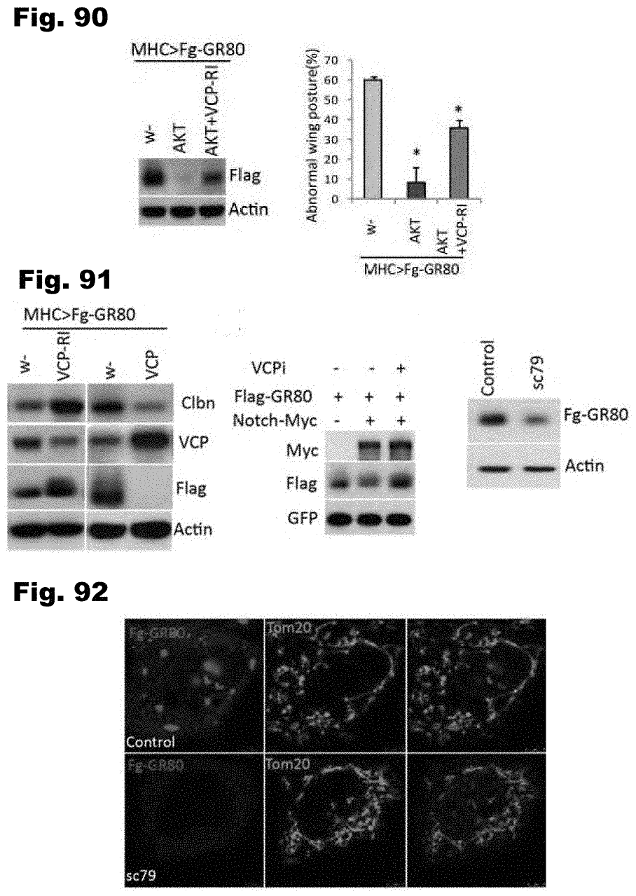

By manipulating individual RQC components, it was found that VCP had the most prominent effect in blocking the effect of Notch on GR80 (FIG. 88), suggesting that VCP is a key mediator of Notch. Since VCP protein level was not affected by Notch (FIG. 89), the possibility of post-translational modification of VCP by non-canonical Notch signaling was considered, and thus the potential role of AKT was assessed. A robust physical interaction between AKT and VCP was found (FIG. 89). Chemical activation of AKT increased VCP phosphorylation at consensus AKT-target site(s), whereas inhibition of AKT had opposite effect (FIG. 89), suggesting that VCP is a genuine AKT substrate. Consistent with VCP being a key downstream effector of AKT, VCP RNAi also attenuated the inhibitory effect of AKT on GR80 (FIG. 90). In addition to VCP, Clbn was also regulated by Notch signaling. Clbn protein level was significantly increased in GR80 fly muscle, an effect abolished by Notch or AKT (FIGS. 84 and 89). Although the detailed mechanisms of Clbn upregulation by GR80 and its subsequent down-regulation by Notch/AKT remain to be delineated, it appeared that VCP was involved (FIG. S4D). Thus through activation of VCP and inhibition of Clbn, non-canonical Notch prevents the build-up of CAT-tailed poly(GR).

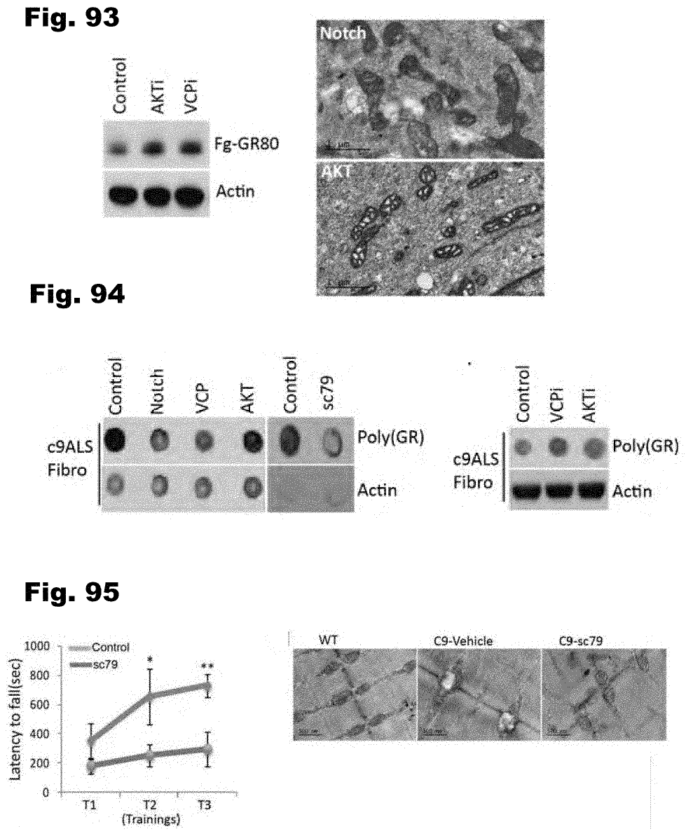

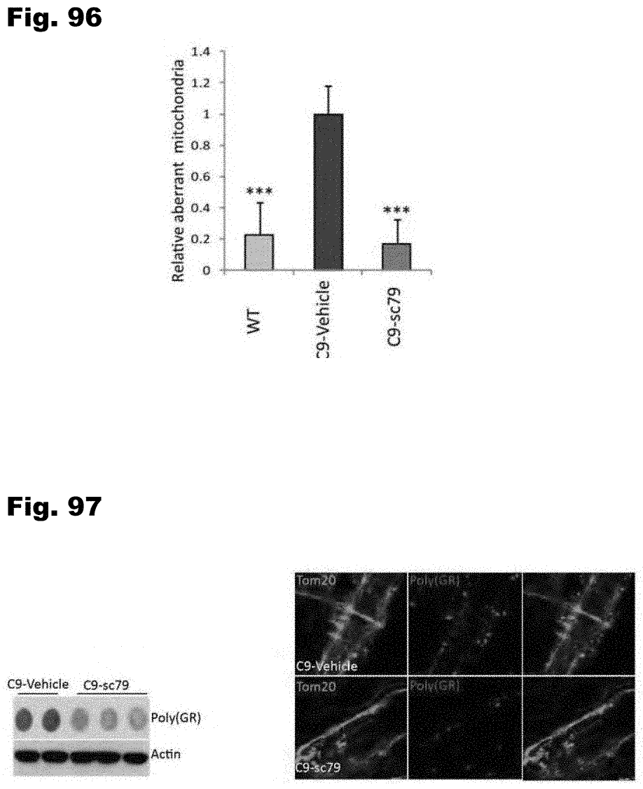

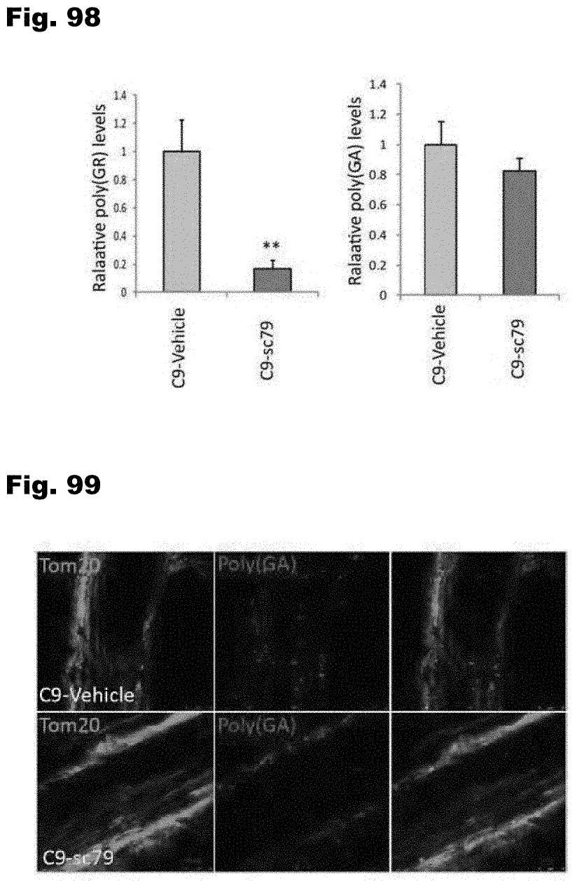

Non-canonical Notch regulation of poly(GR) was then tested in mammalian cells. In HEK293T cells, overexpression of Notch (FIG. 91), or treatment of cells with the ATK activator SC79 (FIGS. 91 & 92) reduced Flag-GR80 level, whereas small molecule inhibitor of AKT (AKTi) or VCP (VCPi) increased Flag-GR80 level (FIG. 93). Similar to fly tissue, the Notch overexpression effect was blocked by VCPi (FIG. 91). In patient fibroblasts, overexpressing non-canonical Notch pathway genes (Notch, AKT) or RQC factor VCP, or SC79 treatment significantly reduced poly(GR) level (FIG. 94) and rescued mitochondrial morphology (FIG. 93). On the other hand, AKTi or VCPi increased poly(GR) level (FIGS. 78 & 94). To further test the in vivo relevance, the C9ALS/FTD BAC transgenic mice (C9-500) were examined, which express a human C9orf72 gene with ˜500 G4C2 repeats. Treatment of C9-500 mice by i.p. injection of SC79 (40 mg/kg/day) improved motor behavior as detected with the rotarod assay (FIG. 95). This was correlated with rescue of muscle mitochondrial morphology (FIGS. 95 & 96) and reduction of poly(GR) (FIGS. 97 & 98) but not poly(GA) (FIGS. 98 & 99) levels in muscle tissue. The regulation of poly(GR) expression by non-canonical Notch signaling therefore is a common theme in C9ALS/FTD models.

Proteostasis failure increases with aging and is a common feature of age-related diseases. How it originates in the disease process is not well defined. Quality control starts when NPCs are still associated with ribosomes. It was found that translation of poly(GR) frequently leads to ribosome stalling, consistent with report of translational stress associated with poly(GR). Ribosome-stalled poly(GR) on mitochondrial surface was detected, and inefficient resolution of this defect led to accumulation of CAT-tailed poly(GR), which can enter mitochondria or form protein aggregates to cause damage. Ribosome-stalled GR80 may also sequester RQC factors away from their normal substrates, creating a deficiency of cellular RQC function, which may lead to neurodegeneration. Two mechanisms were identified, mediated by non-canonical Notch signaling and YME1L, in the quality control of poly(GR) at the co-translational and post-translational levels. Future studies will explore possible interplay between these two mechanisms. Besides identifying poly(GR) as the first CAT-tailed disease-associated protein, this study further emphasizes the essential role of RQC to mitochondrial homeostasis. Other important issues regarding the role of RQC/CAT-tailing in ALS/FTD remain: The crosstalk between RQC/CAT-tailing and mitochondrial signaling; the general role of mitochondrial dysfunction in ALS/FTD; the relationship between RQC/CAT-tailing and stress granule formation; and the effect of RQC/CAT-tailing in neuronal settings.

### Puromycin Labeling of Stalled NPCs

HEK293T cells transfected with pcDNA-Flag-GR80 were treated with puromycin (100 μM, Sigma) and emetine (200 μM, Sigma) at 37° C. for 5 min before harvesting. Cells were then placed on ice, washed with cold HBS, and subjected to mitochondrial purification. To preferentially label stalled NPCs, cells were treated with homoharringtonine (HTT) (5 μM, Tocris Bioscience) for 10 min to prevent new translation initiation and allow active ribosomes to run off before the addition of puromycin/emetine. NPCs on MOM were subjected to western blot or immunoprecipitation analysis. For in vitro puromycin labeling of NPCs on MOM, purified mitochondria were suspended in 10 mM Tris (pH 7.4), 400 mM KCl, 3 mM MgCl₂, and 2 μM biotin-linked puromycin (Jena Bioscience). Puromycylation reactions were performed at 37° C. for 90 min, and postreaction, biotin-puromycin labeled NPCs were purified with Pierrce™ Neutravidin™ agarose beads and subjected to further analysis.

### Mouse Studies

The C9-500 BAC transgenic mouse line expressing a human C9orf72 gene with ˜500 G4C2 repeats were ordered from Jackson Laboratory. The mice were bred and housed in our institutional animal facility. All mouse experiments were approved by our institutional Administrative Panel on Laboratory Animal Care (APLAC) Committee under a designated protocol.

Female C9-500 BAC transgenic mice were i.p. injected with sc79 (Selleckchem) at 40 mg/kg/day (n=10) or vehicle (DMSO [Sigma]: PEG300 [Spectrum]: 2-Hydroxypropyl-beta-cyclodextran (20%) [Sigma] at 1:3:1) (n=8) from 16 weeks of age and continued for 3 months. After stop dosing for 2 weeks, mice were put on the rotarod machine (ENV-574M) to test their ability to move forward and keep their balance. The rod was initially rotating at 16 rpm constant speed to allow positioning of all the mice in their respective lanes. Once all the mice were “ready”, the start button was pushed and the test began. The animals were first trained for 3 days at 5 minutes/day. Then the machine was set up to accelerate from 4 rpm to 40 rpm for testing motor function in the following 3 days for 800 sec/day. The fall-off times of each animal were recorded and used for statistical analysis.

For immunostaining, transgenic C9-500 females (with or without sc79 treatment) and age-matched (39 weeks) WT female mice were sacrificed. Tibialis anterior (TA) muscles were dissected and fixed in 1% paraformaldehyde at 4° C. overnight. The next day myofibers were torn into 10-20 thin muscle bundles (5 to 20 fibers per bundle). Samples were immunostained with anti-Tom20 (Santa Cruz, sc17764) and anti-poly(GR) (Millipore, MABN778), or anti-poly(GA) (Proteintech, 24492-1-AP). Samples were rinsed for 3 times in PBS with 0.1% Triton-X100 for 1 hour each and then stained using secondary antibodies. Confocal images were taken at 40× magnification.

### DOCTRINE OF EQUIVALENTS

In particular, as can be inferred from the above discussion, the above-mentioned concepts can be implemented in a variety of arrangements in accordance with embodiments of the invention. Accordingly, although the present invention has been described in certain specific aspects, many additional modifications and variations would be apparent to those skilled in the art. It is therefore to be understood that the present invention may be practiced otherwise than specifically described. Thus, embodiments of the present invention should be considered in all respects as illustrative and not restrictive.

1. A method of treating a subject having expansion of G4C2 repeats in C9ORF72 comprising:
administering to a subject a noncanonical Notch signaling agonist, an AKT agonist, a YME1L agonist, an inhibitor of CAT-tailing, a mitochondrial uncoupler, or a potassium ion-proton antiporter.

2. The method of claim 1, wherein the subject is a human, an animal model, a cell line model, an organoid model, or a tissue model.

3. The method of claim 1, wherein the subject is a human having amyotrophic lateral sclerosis or frontotemporal degeneration.

4. The method of claim 1, wherein the subject is a human having Alzheimer's disease or Parkinson's disease.

5. The method of claim 1, wherein the subject is administered a noncanonical Notch signaling agonist or an AKT agonist selected from: SC79, kinetin, kinetin triphosphate, kinetin monophosphate, kinetin riboside, or phosphatidylinositol (3,4,5)-triphosphate (PIP3).

6. The method of claim 1, wherein the subject is administered a YME1L agonist.

7. The method of claim 1, wherein the subject is administered a CAT-tailing inhibitor selected from: anisomycin, puromycin, neomycin, borrelidin, or alanyl-AMP.

8. The method of claim 1, wherein the subject is administered a mitochondrial uncoupler selected from: 2, 4-dinitrophenol (DNP); DNP-methylethyl (DNPME), diazoxide, Carbonyl cyanide 4-(trifluoromethoxy) phenylhydrazone (FCCP), Carbonyl cyanide 3-chlorophenylhydrazone (CCCP), 4-[(E)-2-(5,6,7,8-Tetrahydro-5,5,8,8-tetramethyl-2-naphthalenyl)-1-propenyl]benzoic acid (TTNPB), valinomycin; N5,N6-bis(2-Fluorophenyl)-oxadiazolo-pyrazine-5,6-diamine (BAM15), controlled-released mitochondrial protonophore (CRMP), Rhodamine 19 butyl ester (C4R1), Dodecyltriphenylphosphonium (C12TPP), niclosamide ethanolamine salt (NEN), or 10-(6′-plastoquinonyl) decyltriphenyl-phosphonium) (SkQ1).

9. The method of claim 1, wherein the subject is administered a K⁺/H⁺ antiporter selected from: nigericin, monensin, narasin, salinomycin, lenoremycin, septamycin, carriomycin, X-14766A, noboritomycin A, alborixin, ionomycin, A23187, X=14547A, lysocellin, or lasalocid.

10. A method of treating a subject having expansion of G4C2 repeats in C9ORF72 comprising:
administering to the subject a genetic modifier to increase noncanonical Notch signaling agonist.

11. The method of claim 10, wherein the genetic modifier increases expression of Notch, PINK1, mTORC2, or AKT.

12. The method of claim 10, wherein the subject is a human, an animal model, a cell line model, an organoid model, or a tissue model.

13. A method of treating a subject having expansion of G4C2 repeats in C9ORF72 comprising:
administering to the subject a genetic modifier to increase YME1L expression.

14. The method of claim 13, wherein the subject is a human, an animal model, a cell line model, an organoid model, or a tissue model.

15.-24. (canceled)
